(19) United States
(12) Patent Application Publication
(10) Pub. No.: US 2025/0025301 A1
(43) Pub. Date: Jan. 23, 2025

(54) TRANSCATHETER ARTIFICIAL CUSP FOR VALVE INSUFFICIENCY

(71) Applicant: Cuspa Ltd., Nazareth (IL)

(72) Inventors: Ariel WEIGLER, Haifa (IL); Shalom HAZAN, Yardena (IL)

(73) Assignee: Cuspa Ltd., Nazareth (IL)

(21) Appl. No.: 18/714,628

(22) PCT Filed: Nov. 29, 2022
(86) PCT No.: PCT/IL2022/051271

§ 371 Date: May 30, 2024

Related U.S. Application Data
(60) Provisional application No. 63/285,098, filed on Dec. 2, 2021.

(51) Int. Cl. A61F 2/24

(52) U.S. Cl. CPC A61F 2/246; A61F 2/2466; A61F 2210/0014; A61F 2220/0008

(57) ABSTRACT

An obstructing device mountable on a heart valve cusp or leaflet, comprising:
an hollow tubular member, comprising:
an attachment element configured to trap said leaflet or cusp comprising:
   a peripheral frame having a distal end, a proximal end and two side arms;
   a clipping arm extending distally from said proximal end, located between said side arms and having a free end, so that said cusp or leaflet can be trapped between said frame and said clipping arm;
wherein said hollow tubular member is attached to said peripheral frame;
wherein said peripheral frame and said central arm are each elastically deformable and have a preferred bending plane to apply clamping force towards said leaflet or cusp when said leaflet or cusp is trapped therebetween; and
wherein said peripheral frame and said central arm are each curved in their respective preferred bending plane.

### RELATED APPLICATION/S

This application claims the benefit of priority of U.S. Provisional Patent Application No. 63/285,098 filed on Dec. 2, 2021, the contents of which are incorporated herein by reference in their entirety.

### FIELD OF THE INVENTION

The present invention relates to the field of medical devices. More particularly, the present invention, in some embodiments thereof, relates to an artificial cusp for trans-catheter treatment of valve insufficiency.

### BACKGROUND OF THE INVENTION

Valvular insufficiency is a result of valve leakage when the valve is in a closed state. Four main valvular insufficiencies are Aortic regurgitation (AR) also known as aortic insufficiency, mitral regurgitation (MR), tricuspid regurgitation (TR), and pulmonary regurgitation (PR). The main treatment for these pathologies, when they are in a state causing symptoms or significant cardiac remodeling, is valve replacement through open heart surgery, or in some cases catheter-based therapy. However, these treatments are limited to MR only, and TR, under specific conditions.

For example, the aortic valve lies between the left ventricle and the aorta. When the left ventricle contracts during each heartbeat (systole), pressure rises in the left ventricle. When the pressure in the left ventricle rises above the pressure in the aorta, the aortic valve opens, allowing blood to exit the left ventricle into the aorta. The left ventricle actually pushes blood through three flexible cuplike leaflets which make up the aortic valve. When the left ventricle relaxes (when ventricular systole ends) pressure in the left ventricle rapidly drops and the aortic pressure forces the aortic valve to close. The aortic valve closes and prevents blood from flowing back to the left ventricle.

However, in the case of AR, valve leakage occurs when the aortic valve is in a closed state. The leaflets partially close in the aortic valve in the close state, leaving a portion of the aortic valve opened (regurgitant orifice), what causes a portion of the blood to flow back into the left ventricle. This necessitates the heart to work harder, causing a deterioration to the health of a patient. FIG. 1A shows an example of a healthy normal heart where the aortic valve is fully closed. FIG. 1B shows an example of AR with an abnormal aortic valve which fails to fully close allowing the blood to leak backwards into the left ventricle.

Similar leakages occur to the mitral (bicuspid) valve, the tricuspid valve and the pulmonary valve in cases of MR, TR and PR, respectively, mutatis mutandis.

The current standard of care treatment is valve replacement performed by invasive open heart surgery accompanied by severe trauma to the patient and long rehabilitation time.

US 2015/0230919 A1 Describes a method for leaflet prolongation of the mitral or tricuspid valve. The device is implanted around the leaflet therefore extending it and potentially closing the malcoaptation gap.

However, such prior art approaches may be highly invasive and risky procedures. The heart, in prior art approaches based on implantations, may be non-tolerant to the implantation positions. An improved efficient closure of the gap may be useful.

WO 2020026234 relates to an obstructing device comprising a hollow tubular member comprising: a. a proximal opening at its proximal end; b. a substantially tubular surface extending distally from said proximal opening; c. a distal end. The obstructing device further comprises a grasping arm extending distally from said opening at the proximal end; wherein the distal end is either closed or comprises a small orifice. This publication also relates to a method of delivery of the device.

U.S. Pat. No. 9,414,918 relates to prosthetic devices and related methods for helping to seal native heart valves and prevent or reduce regurgitation therethrough, as well as devices and related methods for implanting such prosthetic devices.

An improved and efficient means for closing the gap and a structure for its easy attachment and implantation may be useful. Optionally or additionally, an improved easy method for its delivery, attachment and/or implantation may be useful.

### SUMMARY OF THE INVENTION

It is an object of some embodiments of the present invention to provide a method and means for preventing or reducing blood leakages in cases of AR, MR, TR and PR.

It is an object of some embodiments of the present invention to provide an easily attachable device that prevents the aforementioned blood leakages.

It is an object of some embodiments of the present invention to provide an easy method of delivery thereof.

Other objects and advantages of the present invention may become apparent as the description proceeds.

Aortic regurgitation also known as aortic insufficiency is a condition where the aortic valve fails, allowing backflow of blood from the aorta into the left ventricle of the heart. The present invention, in some embodiments thereof, relates to an obstructing device mountable on to one of the native aortic cusps which reduces the back flow to a minimum. An exemplary delivery method is a minimally invasive transcatheter procedure through the groin whereby a crimped device is provided inside a delivery system and is advanced over a guidewire into the left ventricle. As it is placed near the native valve, the device is deployed out of the delivery system and mounted on the desired cusp. With the assistance of fluoroscopy and ultrasound imaging (and/or a two-way steering mechanism) the delivery system is advanced towards the native cusp and the device deployed and mounted on the native cusp. The device “clips on” to the native cusp for perfect attachment. After deployment and retraction of the delivery system the device remains attached to the native cusp and the regurgitation is significantly reduced. Thanks to some embodiments of the present invention, patients can look forward to a shorter recovery time regaining quality of life.

The present invention obstructing device, in some embodiments thereof, enables grasping the native cusp in its center, in a secure, yet relatively atraumatic attachment. Thus, potentially preserving normal cusp motion. The present invention artificial cusp, in some embodiments thereof, being at the aortic valve expands during diastole and collapses during systole, significantly reducing backflow while minimally interfering with forward flow.

The present invention, in some embodiments thereof, relates to a device for alleviating valvular insufficiency. The present invention, in some embodiments thereof, relates to the treatment of valvular insufficiency by attaching an artificial valve cusp to a native or artificial cusp or leaflet. By doing this it improves heart function by preventing or reducing valve leakage. The artificial cusp is collapsible to prevent valve stenosis when the valve is in an open state.

The present invention, in some embodiments thereof, relates to an obstructing device comprising a hollow tubular member for sealing the regurgitant orifice when the respective heart valve is closed and an attachment element attached to said hollow tubular member. A relatively wide opening/dimension of the hollow tubular member substantially fills and blocks the regurgitant orifice when the heart valve is in a closed state thus reducing or preventing blood leakage. The hollow tubular member opening faces the “leaking blood” such that the leaking blood enters the hollow tubular member via said opening.

The attachment element, in some embodiments thereof, is attachable to the respective native cusp/leaflet. Optionally, the attachment element comprises material having a shape memory effect. It is manufactured by shape setting to have an arc shape when undeformed (in room temperature). The attachment element comprises two main portions which enable it to function as a clip and attach itself to a native cusp like a clip. The first main portion is a peripheral frame with a hollow interior and optionally forms a closed loop. The second main portion is a central arm extending from one edge of the peripheral frame and having a free end. Both the peripheral frame and the central arm optionally curve similarly in the same direction and have an arc shape when undeformed. The attachment element is mounted on the native cusp by initially having the peripheral frame in a “deformed” straightened position while the central arm has an arc shape when undeformed. The native cusp/leaflet is placed within a gap formed between the curved central arm and the surface of the straightened peripheral frame. Thereafter, the peripheral frame is released to return to its arc shape when undeformed, thereby closing the “clip” and being mounted on the cusp/leaflet. The central arm and peripheral frame each curve engaging an opposite side of the cusp/leaflet thereby providing a robust attachment. The central arm engages one side of the cusp/leaflet while the peripheral frame engages its opposite side.

The present invention, in some embodiments thereof, also relates to a delivery system and to a method of insertion using the delivery system. In some embodiments of the invention, a full evaluation of the aortic valve is performed using a CT scan (“Multiphase evaluation of the aortic valve” protocol) and Transthoracic Echocardiography. Also in the deployment stage, the obstructing device is deployed under fluoroscopy and Transesophageal Echocardiography guidance.

The present invention, in some embodiments thereof, relates to an obstructing device mountable on a heart valve cusp or leaflet comprising:
a hollow tubular member comprising:
   a proximal opening at its proximal end;
   a substantially tubular surface extending distally from said proximal opening;
   a distal end, being either closed or comprising a small orifice;
an attachment element comprising:
   a peripheral frame having a distal end, a proximal end and two side arms, each of said side arms being attached to said distal end and proximal end;
   a central clipping arm extending distally from said proximal end and having a free end;
wherein said hollow tubular member is attached to said peripheral frame; and
wherein said peripheral frame and central clipping arm are both curved and have an arc shape when undeformed.

Preferably, the peripheral frame comprises a hollow interior.

Preferably, the hollow tubular member is attached to the peripheral frame at the exterior side of the arc shape.

Preferably, the distance between the proximal opening and the attachment element proximal end is between 0.1 and 0.5 mm; and wherein the distance between the hollow tubular member distal end and the attachment element distal end is between 0.1 and 0.5 mm.

Preferably, the central clipping arm comprises a plurality of sharp edges at its sides.

Preferably, each one of the side arms comprises a plurality of sharp edges at its inner side.

Preferably, the sharp edges curve backwards or forwards.

Preferably, the peripheral frame distal end and peripheral frame proximal end are each placed at the edges of the arc shape of the peripheral frame; and wherein the central clipping arm free end and the proximal end of the central clipping arm are each placed at the edges of the arc shape of the central clipping arm.

Preferably, the hollow tubular member comprises a membrane.

Preferably, the membrane is self-expandable.

Preferably, the obstructing device further comprising a frame comprising one or more wires;

wherein the membrane is mounted on said frame.

Preferably, the hollow tubular member tappers distally.

Preferably, the peripheral frame comprises one or more apertures.

Preferably, the attachment element comprises material selected from the group consisting of metal alloy and shape memory alloy.

Preferably, the attachment element comprises material selected from the group consisting of nitinol, stainless steel, and cobalt chromium.

Preferably, the attachment element comprises connecting elements.

Preferably, the connecting elements are selected from the group consisting of biocompatible needles, biocompatible pins and biocompatible spikes.

Preferably, the connecting elements slant from the attachment element.

The present invention, in some embodiments thereof, relates to a delivery system for delivering an obstructing device for being mounted on a native cusp or leaflet, said delivery system comprising:
a distal holder comprising:
   a distal front member
   a ring-shaped element extending proximally from said distal front member;
   a top (preferably flat) surface;
a proximal holder comprising:
   a distal surface;
   a lock spring element comprising a spring mechanism configured to push the distal portion of said lock spring element away from the central axis of the proximal holder, and
   wherein a gap is formed between the distal portion of said lock spring element and a portion of said distal surface;
a wire lumen connected to a handle and distally and proximally movable by said handle;
a proximal lumen connected to said handle;
an outer sheath distally and proximally movable by said handle, configured to cover the proximal holder and part of the distal holder and engageable with the distal holder ring-shaped element;
wherein said wire lumen passes through said proximal lumen; wherein said wire lumen is fixedly connected to said distal holder; and
wherein said proximal lumen is fixedly connected to said proximal holder.

Preferably, the delivery system comprises a distal holder internal bore passing through the distal holder and a proximal holder internal bore passing through the proximal holder; wherein the wire lumen passes through said distal holder internal bore and through said proximal holder internal bore.

Preferably, the lock spring element comprises one or more teeth elements extending downwards from the distal portion of the lock spring element.

Preferably, the system further comprises an obstructing device mountable on a heart valve cusp or leaflet comprising:
a hollow tubular member comprising:
   a proximal opening at its proximal end;
   a substantially tubular surface extending distally from said proximal opening;
   a distal end, being either closed or comprising a small orifice;
an attachment element comprising:
   a peripheral frame having a distal end, a proximal end and two side arms, each of said side arms being attached to said distal end and proximal end;
   a central clipping arm extending distally from said proximal end and having a free end;
wherein said hollow tubular member is attached to said peripheral frame; and
wherein said peripheral frame and central clipping arm are both curved and have an arc shape when undeformed;
wherein the peripheral frame distal end is placed within the ring shaped element; and
wherein the peripheral frame proximal end is placed within the gap.

The present invention, in some embodiments thereof, relates to a method for implanting an obstructing device on a heart valve cusp or leaflet, comprising:
creating an opening in a blood vessel;
inserting an introducer sheath;
inserting a guide wire through the introducer sheath and passing it through the blood vessel all the way to the heart valve and therethrough to the respective heart chamber;
providing a delivery system comprising:
   the obstructing device as explained herein;
   a distal holder retaining the peripheral frame distal end;
   a proximal holder retaining the peripheral frame proximal end;
   a wire lumen connected to a handle and distally and proximally movable by said handle, and fixedly attached to said distal holder;
   a proximal lumen connected to said handle and fixedly attached to said proximal holder;
   wherein said wire lumen passes through said proximal lumen;
   an outer sheath distally and proximally movable by said handle, configured to cover the proximal holder and part of the distal holder, wherein the central clipping arm is deformed by being at least partially straightened by the outer sheath restricting it;
passing the delivery catheter system distally by passing the wire lumen over the guidewire until the distal portion of the outer sheath arrives near the respective heart valve cusp or leaflet;
retrieving the outer sheath proximally to an extent where the central clipping arm curves to its arc shape when undeformed;
positioning the obstructing device in the correct intended position to be attached to the native cusp or leaflet;
forwarding the wire lumen distally while the proximal lumen remains stationary thereby releasing the peripheral frame distal end from being retained by the distal holder, and thereby having the peripheral frame curve to its arc shape when undeformed and thereby having the central clipping arm placed tightly engaging the belly of the cusp while the peripheral frame tightly engages the cusp flow facing side;
retrieving the outer sheath proximally to an extent where the peripheral frame proximal end is released from being retained by the proximal holder, thereby releasing the obstructing device to be completely deployed;
proximally retrieving the outer sheath, the distal holder and proximal holder;
proximally retrieving the guide wire;
removing the introducer sheath;
closing the blood vessel.

Preferably, the retrieving of the outer sheath proximally to an extent where the peripheral frame proximal end is released from being retained by the proximal holder, comprises retrieving the outer sheath to an extent such that a lock spring element is released thereby releasing the peripheral frame proximal end from being retained by the proximal holder.

The present invention, in some embodiments thereof, relates to a method for implanting an obstructing device on a heart valve cusp or leaflet, comprising:
creating an opening in a blood vessel;
inserting an introducer sheath;
inserting a guide wire through the introducer sheath and passing it through the blood vessel all the way to the heart valve and therethrough to the respective heart chamber;
providing a delivery system comprising:
   an obstructing device comprising:
   a hollow tubular member comprising:
      a distal opening at its distal end;
      a substantially tubular surface extending proximally from said distal opening;
      a proximal end, being either closed or comprising a small orifice;
   an attachment element comprising:
      a peripheral frame having a distal end, a proximal end and two side arms, each of said side arms being attached to said distal end and proximal end;
      a central clipping arm extending proximally from said distal end and having a free end;
   wherein said hollow tubular member is attached to said peripheral frame; and
   wherein said peripheral frame and central clipping arm are both curved and have an arc shape when undeformed;
   a distal holder retaining the peripheral frame distal end;
   a proximal holder retaining the peripheral frame proximal end;
   a wire lumen connected to a handle and distally and proximally movable by said handle, and fixedly attached to said distal holder;
   a proximal lumen connected to said handle and fixedly attached to said proximal holder;
   wherein said wire lumen passes through said proximal lumen;
   an outer sheath distally and proximally movable by said handle, configured to cover the proximal holder and part of the distal holder, wherein the central clipping arm is deformed by being at least partially straightened by the outer sheath restricting it;
passing the delivery catheter system distally by passing the wire lumen over the guidewire until passing the respective heart valve cusp or leaflet;
retrieving the outer sheath proximally to an extent where the central clipping arm curves to its arc shape when undeformed;
positioning the obstructing device in the correct intended position to be attached to the native cusp or leaflet;
forwarding the wire lumen distally while the proximal lumen remains stationary thereby releasing the peripheral frame distal end from being retained by the distal holder, and thereby having the peripheral frame curve to its arc shape when undeformed and thereby having the central clipping arm placed tightly engaging the belly of the cusp while the peripheral frame tightly engages the cusp flow facing side;
retrieving the outer sheath proximally to an extent where the peripheral frame proximal end is released from being retained by the proximal holder, thereby releasing the obstructing device to be completely deployed;
proximally retrieving the outer sheath, the distal holder and proximal holder;
proximally retrieving the guide wire;
removing the introducer sheath;
closing the blood vessel.

Following is a non-exclusive list including some examples of embodiments of the invention. The invention also includes embodiments which include fewer than all the features in an example and embodiments using features from multiple examples, also if not expressly listed below.

Example 1. An obstructing device mountable on a heart valve cusp or leaflet, in a valve lumen having a flow axis between an upstream direction and a downstream direction, comprising:
an hollow tubular member, comprising:
an attachment element configured to trap said leaflet or cusp comprising:
   a peripheral frame having a distal end, a proximal end and two side arms;
   a clipping arm extending distally from said proximal end, located between said side arms and having a free end, so that said cusp or leaflet can be trapped between said frame and said clipping arm;
wherein said hollow tubular member is attached to said peripheral frame;
wherein said peripheral frame and said central arm are each elastically deformable and have a preferred bending plane to apply clamping force towards said leaflet or cusp when said leaflet or cusp is trapped therebetween; and
wherein said peripheral frame and said central arm are each curved in their respective preferred bending plane.

Example 2. An obstructing device according to example 1, wherein said peripheral frame and said central arm are curved in an arc shape in a resting state thereof.

Example 3. An obstructing device according to example 1, wherein the hollow tubular member is attached to the peripheral frame at the exterior side of the arc shape thereof.

Example 4. An obstructing device according to example 2 or example 3, wherein said arc shape of said frame extends from said proximal end to said distal end thereof and wherein said arc shape of said clipping arm extends from a proximal end to a distal end.

Example 5. An obstructing device according to any of the preceding examples, wherein said tubular member is elongate and has an axis and wherein said attachment element is elongate and has an axis and wherein said elongate hollow tubular member is attached to said peripheral frame such that when said elongate attachment element is mounted on a cusp so that said axis points in a downstream direction, said elongate hollow tubular member also points in a downstream direction.

Example 6. An obstructing device according to any of the preceding examples, wherein said hollow tubular member comprises:
a proximal opening at its proximal end;
a substantially tubular surface extending distally from said proximal opening;
a distal end, being either closed or comprising a small orifice;
Example 7. An obstructing device according to any of the preceding examples, wherein in said peripheral frame, each of said side arms being attached to said distal end and proximal end and said clipping arm does not extend to said distal end.

Example 8. The obstructing device according to any of the preceding examples, wherein the peripheral frame comprises a hollow interior through which said clipping arm can pass absent said leaflet or cusp.

Example 9. The obstructing device according to any of the preceding examples, wherein the distance between the proximal opening and the attachment element proximal end is between 0.1 and 0.5 mm; and wherein the distance between the hollow tubular member distal end and the attachment element distal end is between 0.1 and 0.5 mm.

Example 10. The obstructing device according to any of the preceding examples, wherein the clipping arm comprises a plurality of protrusions adapted to engage cusp tissue and extending in a direction away from said preferred bending plane.

Example 11. The obstructing device according to any of the preceding examples, wherein each one of the side arms comprises a plurality of protrusions adapted to engage cusp tissue and extending in a direction towards the other side arm.

Example 12. The obstructing device according to any one of examples 10 or 11, wherein the sharp edges curve in a proximal or a distal direction.

Example 13. The obstructing device according to any of the preceding examples, wherein the hollow tubular member comprises a membrane.

Example 14. The obstructing device according to example 13, wherein the membrane is self-expandable.

Example 15. The obstructing device according to any one of examples 13 or 14, further comprising a frame comprising one or more wires; wherein the membrane is mounted on said frame.

Example 16. The obstructing device according to any of the preceding examples, wherein the hollow tubular member tappers distally in a downstream direction.

Example 17. The obstructing device according to any of the preceding examples, wherein the peripheral frame comprises one or more apertures.

Example 18. The obstructing device according to any of the preceding examples, wherein the attachment element is made of a super-elastic or shape memory material.

Example 19. The obstructing device according to any of the preceding examples, wherein the attachment element comprises connecting elements configured to engage cusp material.

Example 20. The obstructing device according to example 19, wherein the connecting elements slant from the attachment element.

Example 21. The obstructing device according to any of the preceding examples, wherein the clipping arm includes at least one cusp engaging needle extending in a preferred bending plane thereof.

Example 22. The obstructing device according to any of the preceding examples, wherein in said resting state, a gap is defined between the frame and clipping arm, said gap having an opening greater than 6 mm.

Example 23. A delivery system for delivering an obstructing device for being mounted on a native cusp or leaflet, said delivery system comprising:
a body;
a capsule at a distal end of said body, the capsule comprising:
   a recess for receiving an obstructing device;
   a distal holder for holding a distal part of said obstructing device;
   a proximal holder for holding a proximal side of said obstructing device;
wherein said one of said proximal and said distal holders is movable to two positions, a first position where a section of said obstructing device is exposed, allowing part of said obstructing device to elastically extend away from said capsule and a second position where the proximal or distal end of the obstructing device is released from being held.

Example 24. A delivery system according to example 23, comprising a spring configured to push said obstructing device away from said capsule when said obstructing device is released at both a distal end and a proximal end thereof.

Example 25. A delivery system according to example 23 or example 24, comprising an obstructing device in said recess, wherein said obstructing device comprises a curved frame with a curved resting state, wherein said capsule holds said frame to prevent said curved frame from being at said curved resting state.

Example 26. A method for implanting an obstructing device on a heart valve cusp or leaflet, comprising:
advancing passing a delivery catheter system having an obstructing device mounted therein, until the distal portion of the catheter arrives near a target heart valve cusp or leaflet;
releasing a clipping arm of said obstructing device to extend radially away from said delivery catheter system and define a gap therebetween;
positioning said cusp or leaflet within said gap; and
further releasing at least a further part of said obstructing device to elastically extend away from said delivery catheter system so that said gap is closed and said cusp or leaflet is trapped by said clipping arm against said obstructing device.

Example 27. A method according to example 26, further comprising releasing said obstructing device entirely from said delivery catheter system using an elastic recoil.

Example 28. A method according to example 26 or example 27, wherein said further releasing an elastically deformable frame and said clipping arm, wherein said cusp or leaflet prevents said clipping arm from reaching a resting position on an opposite side of said cusp or leaflet.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

The present invention, in some embodiments thereof, is illustrated by way of example in the accompanying drawings, in which similar references consistently indicate similar elements and in which:

FIGS. 1A and 1B illustrate a normal functioning heart aortic valve and an Aortic Regurgitation functioning heart aortic valve respectively.

FIGS. 2A-2D illustrate embodiments of the present invention obstructing device.

FIGS. 2E-2F illustrate embodiments of the present invention obstructing device hollow tubular member.

FIG. 2G illustrates an embodiment of the present invention obstructing device attachment element.

FIG. 2H illustrates an embodiment of the obstructing device with the frame and central clipping arm straightened.

FIG. 2I is an image of a frame including a central arm of an embodiment of the obstructing device mounted on a cusp.

FIG. 2J is an image of a frame including a central arm of an embodiment of the obstructing device in a relaxed state.

FIGS. 3A-3K illustrate an embodiment of the present invention delivery system or portions thereof.

FIGS. 4A-4C illustrate an embodiment of the present invention delivery system at different stages.

FIGS. 5A-5D illustrate different stages of a method according to an embodiment of the present invention.

FIGS. 6A and 6B illustrate an embodiment of the present invention device functioning in different valve states.

FIGS. 7A-7B show an example of heart leakages before and after the implanting of an embodiment of the present invention.

FIG. 8 illustrates an embodiment of the present invention delivery system.

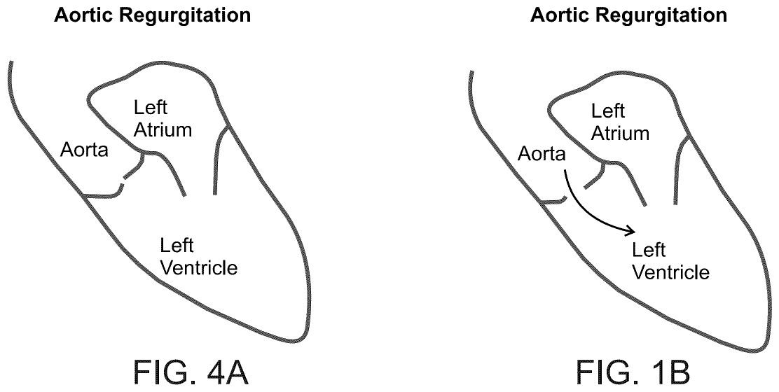

### DETAILED DESCRIPTION OF THE INVENTION

The present invention, in accordance with some embodiments thereof relates to an artificial cusp attachment. More particularly, the present invention relates to a blood leakage obstructing device comprising a hollow tubular member, attachable to one of the heart native valves' (aortic valve, mitral valve, pulmonary valve, tricuspid valve) cusps/leaflets. Potentially, the obstructing device effectively causes the heart native valve to be fully closed when in its closed state. The hollow tubular member can be seen as a scaffold to the cusps/leaflets, an add-on portion that moves with the cusp/leaflet as it opens and closes the valve.

The device of some embodiments of the present invention will be explained mostly in relation to the aortic valve, but may similarly be attached to the other heart native or artificial valves' cusps or leaflets, mutatis mutandis.

In the present specification the “proximal end” refers to the end closest to the medical personnel delivering the device. The “distal end” refers to the end furthest from the medical personnel and closest to the target location in the patient's body during delivery of the device. In relation to the embodiment which inserts the device of the present invention via the aorta (towards the left ventricle), the “distal direction” refers to the direction towards the left ventricle and the “proximal direction” refers to the opposite direction, i.e. the direction towards the aorta away from the left ventricle. Thus, the blood through the aortic valve flows from the distal to the proximal direction.

In cases with hearts having aortic valvular insufficiency, valve leakage occurs when the aortic valve is in a closed state. The leaflets/cusps partially close in the aortic valve in its closed state, leaving a portion of the aortic valve opened, which causes a portion of the blood to flow back into the left ventricle. In accordance with some embodiments of the present invention, the obstructing device which is attached to one of the cusps is situated in the regurgitant orifice of the aortic valve when the aortic valve is in a closed state and effectively obstructs the valvular insufficiency “opening” at the regurgitant orifice of the aortic valve. Thus, the blood from the aorta does not leak back to the left ventricle (but part of the blood only enters the interior of the hollow tubular member of the obstructing device).

The obstructing device of some embodiments of the present invention comprises a hollow tubular member, attachable to the valve cusp. The hollow tubular member of the obstructing device comprises a proximal opened end, a lateral (side) tubular surface and a closed (scaled) distal end or an end provided with a small hole/orifice. More specifically, the obstructing device hollow tubular member comprises a proximal opening at its proximal end, the substantially tubular surface extending distally from the proximal opening and a closed distal end (or an end provided with a small orifice).

The device is such that when attached to the cusp, either:
1. in case where the distal end is completely closed, blood may enter and thereafter exit the interior of the hollow tubular member from the proximal opening only (i.e. the lateral (side) tubular surface along with the distal side is all closed/scaled).
2. in case where the distal end includes a small orifice, blood may enter and thereafter exit the interior of the hollow tubular member mainly from the proximal opening but a very small portion may exit the distal small orifice. However, this minimally affects the function of the heart and most of the potential leakage blood is obstructed by the obstructing device.

The embodiment with the distal end small orifice is potentially advantageous as this configuration prevents clotting of blood within the hollow tubular member by allowing minimal blood flow through the orifice and/or allows opening of the hollow tubular member by blood flow therethrough.

The proximal opening may be placed near the proximal end (at the edge) of the cusp, adjacent to its inner side such that when the aortic valve is in a closed state, the obstructing device obstructs the valvular insufficiency “opening” at the regurgitation orifice of the aortic valve. The hollow tubular member is preferably, but not necessarily, positioned such that it extends up to 3 mm proximally above the proximal edge of the native cusp (the nodule of Arantius).

In some embodiments of the invention, the obstructing device hollow tubular member is compressible and may be expanded such that the volume of its interior may vary. During systole, when the aortic valve is in its open state, the blood exiting the left ventricle at a substantial current flow causes the obstructing device to partially be compressed and its interior volume to thus decrease. During diastole, when the aortic valve is in its closed state, blood from the aorta may enter the obstructing device interior (possibly adding to its expansion thereof and an increase in the obstructing device interior volume). In any case, during diastole, the blood in the aorta does not flow back to the left ventricle as it is blocked by the obstructing device which engages the other cusps flow facing sides (i.e. the sides facing the center of the valve) and effectively forms a seal not allowing (or reducing the ability of) the blood to pass through the engaging locations between the obstructing device and the cusps flow facing sides. The obstructing device effectively causes the heart native valve to be more closed (in some cases-fully closed) when in its closed state, as in the function of a healthy heart. In some embodiments, the present invention obstructing device does not form a total seal (at the regurgitation orifice), but decreases the blood leakage, which also improves the health of a patient.

In some embodiments of the invention, the opening of the hollow tubular member of the obstructing device (at its proximal side) is attached near the cusp proximal end. The obstructing device hollow tubular member engages the cusp flow facing side and extends distally adjacent to and along the cusp that it is attached to (along the cusp flow facing side). Optionally, the obstructing device hollow tubular member tappers distally. In this manner the wide proximal end of the obstructing device hollow tubular member engages the proximal ends of the cusps of the aortic valve (the cusp that it is attached to and the other cusps that their ends tend to close to engage each other) causing the effective seal. The distal part of the obstructing device hollow tubular member attached to the cusp may be narrower than the wide proximal opening, as it is attached to the distal portion of the cusp but does not need to be wide in order to obstruct. This configuration enables the obstructing device to have a minimal mass for a most effective function. The proximal opening is configured to be wide enough to obstruct.

According to an embodiment of the present invention, the obstructing device hollow tubular member is in the form of a deformable membrane. The membrane (in the form of the hollow tubular member) comprises a proximal opening, a lateral tubular surface and either:
1. a closed distal end (preferably tapering distally from the opening along the lateral tubular surface to the distal end). The interior of the membrane is completely closed/scaled (except for the proximal opening).
2. a distal end comprising a small orifice, (the membrane preferably tapering distally from the opening along the lateral tubular surface to the distal end). The interior of the membrane is closed (except for the proximal opening and the distal end orifice).

During systole the membrane may be partially compressed (by the systole blood flow) and during diastole the membrane may expand as the “leakage” blood enters the interior of the hollow tubular member enlarging its volume.

According to one embodiment the membrane is self-expandable (e.g. elastic). During systole the membrane is partially compressed (by the systole blood flow) and during diastole the membrane expands. The membrane may comprise an artificial source, a biocompatible material (e.g. Dacron, PTFE, etc.) or a biologic source (e.g. animal valve cusp, animal pericard, porcine pericardium, bovine pericardium etc.). The membrane may also comprise autologous tissue.

According to another embodiment of the present invention, the obstructing device comprises a frame comprising one or more wires that define its general shape. The wires are structured such that they form a general hollow tubular shape with a closed distal end. The frame wires are structured such that the membrane is attached thereto forming a strengthened hollow tubular member with a proximal opened end, the lateral tubular surface extending distally from the proximal opened end and a closed distal end (optionally with a small orifice). The membrane is actually mounted on the frame (either from within or from without). Preferably the frame shape tappers distally. The present invention may include embodiments with a self expandable hollow tubular member mounted on the wires frame (which may also be self expanding).

In some embodiments of the invention, the obstructing device comprises a clip-like element attached to the hollow tubular member, configured to attach and mount the hollow tubular member to a respective cusp of the heart valve.

An aspect of some embodiments of the invention relates to a cusp-mounted cardiac implant including a frame and one or more arm and in which the frame and/or the arm bend to engage a cusp therebetween. In some embodiments of the invention, the frame has two stable states, each with a different curvature state and wherein in one state, a gap is defined between the arm and the frame and in another state the gap is smaller or negative (the arm goes past the frame. Optionally, the arm is prevented from going past the frame not by the arm extending to or past an edge of the frame, but by the cusp. Some, in some embodiments, one stable state is defined by the existence of a cusp trapped between the arm and the frame. In some embodiments of the invention, the gaps and geometry of the frame and arm and forces applied thereby are selected to avoid or reduce damage to the cusp, other than, for example, penetration of one or more needles into the cusp.

An aspect of some embodiments of the invention relates to a frame for engaging a cusp of a cardiac valve in which the frame is curved. In some embodiments of the invention, the frame extends laterally and axially along a cusp, when engaging the cusp and the curvature is a plane perpendicular to the cusp surface. In some embodiments of the invention, the frame is elastic and is formed of multiple elongate elements, for example, an elongate frame and a clipping arm extending from one side of the frame towards the other side of the frame, with the cusp to be trapped between the frame and the arm. Optionally, one or more or all of the elongate elements define a preferred bending plane, within which resistance to bending is lower. For example, the elongate elements may be laterally flat with a thin and a thick dimension and have a preferable bending plane which extends along the length of such elongate element and in a direction of the thin dimension. All the elongate elements optionally share a same preferred bending plane which is optionally perpendicular to the cusp, when trapped. It is noted that the term “plane” also includes slightly curved surfaces, as the elongate elements may not bend exactly in a flat plane.

A potential advantage of having a curved shape, especially an arc shape, for the frame is that such shape may more closely conform to the shape of a cusp in a closed state of the cardiac valve. This may reduce deformation of the cusp, preventing damage thereto and/or avoiding regurgitation caused by such deformation. Optionally, the curvature is not too great, to prevent pathological blocking of the valve by the cusp in an open state of the valve. For example, the curvature in a deployed state may be, for example between 20 and 200 mm or more, for example, between 30 and 100 mm. Optionally or additionally, the arc size in angle of the frame is between 10 and 60 degrees, for example, between 15 and 30 degrees. It is noted that in a resting state, the frame may be considerably more deformed and/or have a smaller radius of curvature, for example, a radius of curvature which is a factor of 0.5 or 0.25 or more of said radii.

In an obstructing device configuration, according to some embodiments of the invention, the frame has attached thereto a hollow obstructing member, which can be compressed by cardiac flow and expanded to block the valve in a closed state of the valve. In some embodiments of the invention, the member is constrained to be curved by the shape of the frame. Optionally or additionally, the frame is not attached to the member over the entire length of the member, which may allow, for example, for a distal part of the member to extend radially away from the cusp and the frame. A gap defined thin may be, for example, between 1 and 10 mm, for example, between 2 and 6 mm. Optionally or additionally, the member may be curved, at least in part, to conform to a cusp shape.

It is noted that a natural cusp may be flatter at a coapting section thereof where two cusps meet. Optionally, the frame is designed to be flatter at such section than in a more downstream section.

An aspect of some embodiments of the invention relates to a method for engaging a cusp of a cardiac valve using at least three elongate elements on alternating sides of a cusp, where at least one of the elements, optionally all the elements, include one or more projections, optionally a row of projections which lie generally I a plane of the cusp and the elements. In some embodiments of the invention, the elements are arranged into a frame with a central arm and the frame includes serrations projecting towards the arm and the arm includes serrations projecting towards the frame.

In some embodiments of the invention, an additional anchoring is provided using one or more needles (used as a term for penetrating elements) which penetrate into the cusp. Optionally or additionally, non-penetrating elements may be provided, e.g., projections pointed perpendicular form the plane of the cusp and/or the elements.

In some embodiments of the invention, the elements are formed by cutting a sheet, for example, using laser cutting. Alternative, one or more of the elements are formed by a bent wire.

An aspect of some embodiments of the invention relates to a method of delivering a clasping anchoring element to a cardiac cusp. In some embodiments of the invention, the element includes a frame and a central arm, which central arm is optionally attached to the frame on one side thereof but optionally does not reach to the opposite side of the frame.

In some embodiments of the invention, the method includes releasing the central arm to elastically extend away form a delivery system and the frame, thereby defining a gap into which a cusp portion may be positioned.

Optionally, thereafter, a part of the frame is released to close the gap and engage the cusp between the frame and the arm.

Optionally, a remaining portion of the frame is help until final deployment. This potentially allows the anchoring element to be removed from the cusp. Optionally or additionally, the frame has a reduced locking force against the arm until the remaining portion is release and allowed to bend. Optionally, the remaining portion comprises a base which elastically (including via shape memory and super-elastically) interconnects the arm and the frame.

In some embodiments of the invention, a delivery system is used which includes an optional bending ability (e.g., to help align the gap with the cusp). Optionally or additionally, the delivery system provided an ability to move just the tip a short distance, for example, between 0.5 and 3 cm, for example, between 1 and 2 cm, thereby allowing the frame (or a containing capsule) to be moved without repositioning the delivery system as a whole.

Optionally or additionally, the delivery system includes a capsule to hold the frame and a tab to selectively release the arm (e.g., retract a sheath or tab from a side of the frame enough so the arm is released, but not far enough so that the frame is released) and/or the frame (e.g., by further retraction).

Optionally or additionally, the delivery system includes a tab to selectively release the remaining part of the frame when delivery is completed. Optionally, such release is assisted by an elastic element which pushes the frame away from the capsule. Optionally or additionally, such elastic release is provided by the frame itself.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

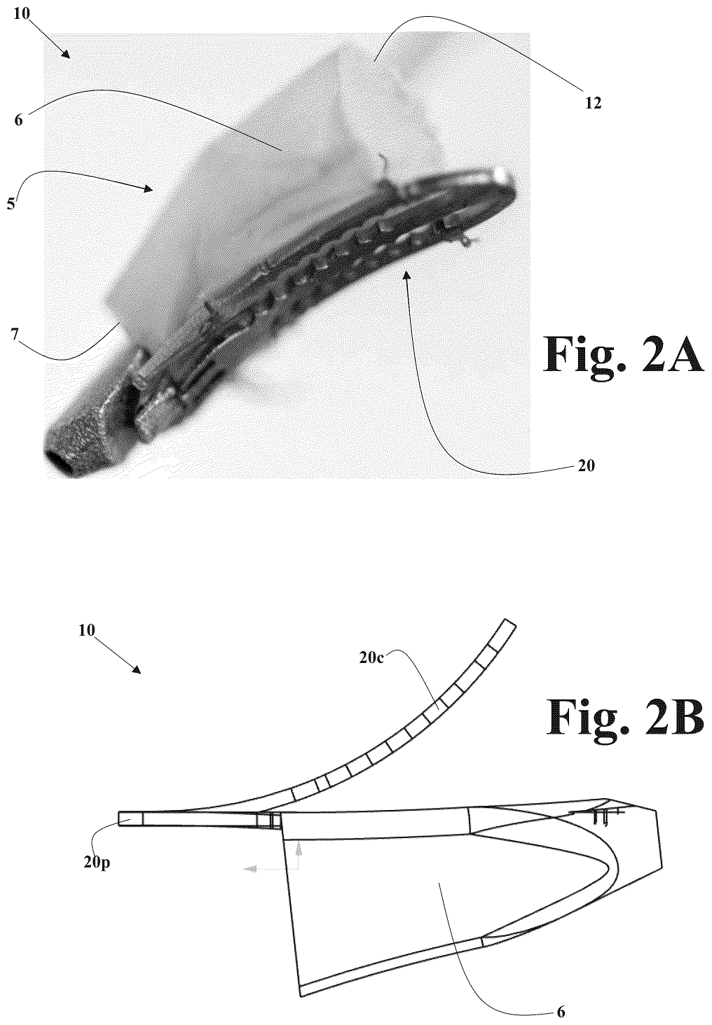

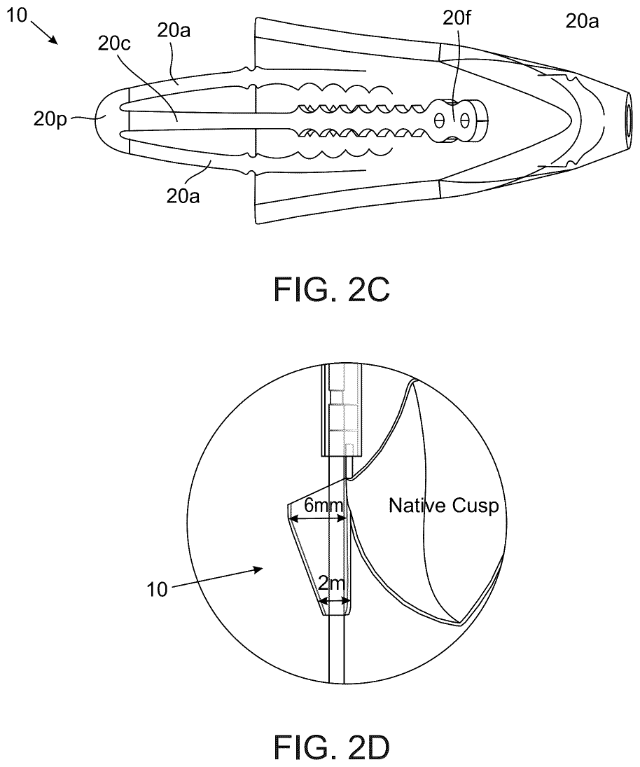

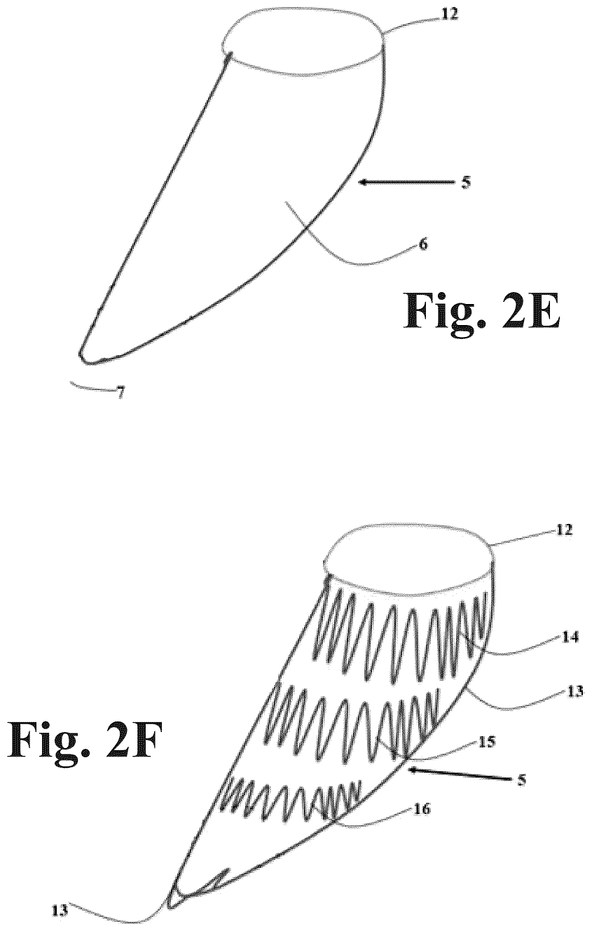

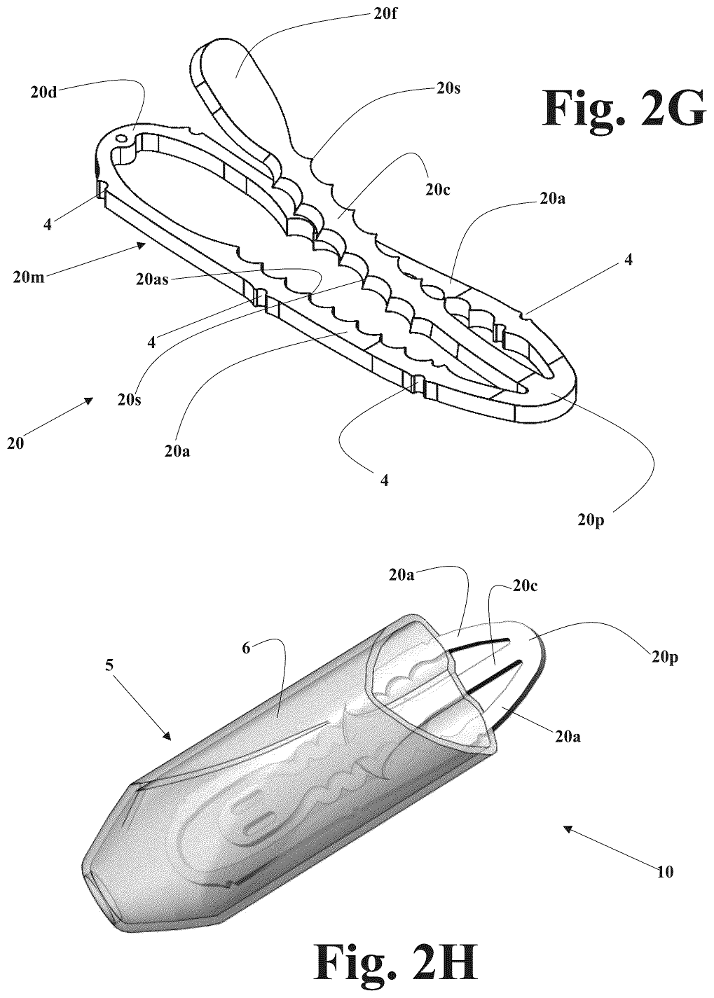

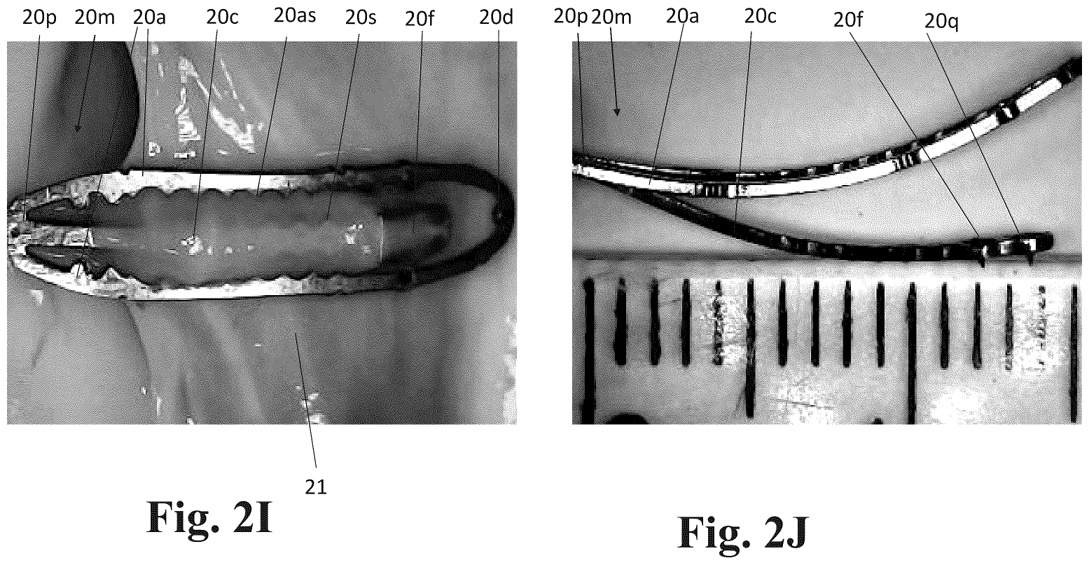

FIG. 2A shows an embodiment of the present invention obstructing device 10. The obstructing device 10 comprises a membrane that forms a hollow tapering tubular member 5 with a proximal opening 12. The interior of tapering tubular member 5 is completely closed/scaled by the membrane (except for the proximal opening 12). A lateral tubular surface 6 extends distally from the proximal opening 12, tappers distally and terminates at the closed distal end 7 (wherein in other embodiments (not shown) the distal end may comprise an orifice/hole). The hollow tubular member 5 is attached to an attachment element 20 (also referred to herein as “attaching element” interchangeably).

FIG. 2E shows an embodiment of the present invention hollow tubular member 5 comprising a frame comprising one or more wires that define its general shape. The membrane of the obstructing device 10 (not shown) is attached to the frame, such that the frame and membrane attached thereto form together a hollow tapering tubular member 5 with a proximal opening 12 (wherein the interior of tapering tubular member 5 is completely closed/scaled by the membrane except for the proximal opening 12 and optionally a distal orifice, as explained herein). In one embodiment the frame is external to the membrane layer (inside or outside the member). In another embodiment, the wires of the frame may be contained within the layer of the membrane. The frame is designed to withstand fatigue, and may be collapsible and/or self-expanding.

According to an embodiment of the present invention, the tapering tubular member 5 comprises a round (preferably circular) wire portion on its proximal side forming the proximal opening 12. The tapering tubular member 5 comprises one or more longitudinal wire elements 13 along its length. The tapering tubular member 5 comprises one or more transverse wire elements (e.g. circular) surrounding one or more respective transverse portions of the tapering tubular member 5 placed along its length.

The embodiment of FIG. 2F shows three transverse surrounding wire portions 14, 15, 16, each in the form of a sinusoidal wave. Since the tapering tubular member 5 tappers distally, in case of each two adjacent transverse surrounding wire portions, the more proximal one of the two is larger (i.e. it surrounds a longer width portion) than the more distal one of the two. In this case, wire portion 14 is optionally greater that wire portion 15, which is optionally greater than wire portion 16.

In some embodiments the tapering tubular member 5 may comprise one or more longitudinal wire elements along its length and one or more transverse wire elements surrounding one or more respective transverse portions of the tapering tubular member 5 placed along its length.

The obstructing device 10 further optionally comprises an attaching element 20. The attaching element alone is shown in FIG. 2G. The attaching element 20 may be in the form of a snap clip, bendy, snap barrette, contour clip. The attaching element 20 comprises a metal alloy (e.g., nitinol) that tends to bend/curve thereby forming an arc. The attaching element 20 comprises two elongated (typically thin) side arms 20a. The attaching element 20 comprises a proximal end 20p, the two side arms 20a extending distally from said proximal end 20p, and a central clipping arm 20c extending distally from said proximal end 20p. The attaching element 20 comprises a distal end 20d attached to the distal portions of the side arms 20a. While the side arms 20a are optionally connected to both the proximal end 20p and the distal end 20d, the central clipping arm 20c is connected only to the proximal end 20p and has a distal free end 20f (i.e., the free end is not connected to the frame distal end 20d). The proximal end 20p, distal end 20d and side arms 20a form a peripheral frame 20m having a hollow interior with the central clipping arm 20c extending distally from the proximal end 20p within the hollow interior.

In FIG. 2G the frame 20m is flat and the central clipping arm 20c is in the form of an arc. This is the situation at a certain stage of the insertion process as explained herein (where the frame is retained in a flat manner and the central clipping arm 20c is released thereby curving into an arc form). It should be born in mind that when the frame 20m is released from being retained, it also curves into an arc typically to the same extent as that of the central clipping arm 20c. The distal end 20d and proximal end 20p are each placed at the edges of the arc shape of peripheral frame 20m. Also, the free end 20f and the attachment point of the central clipping arm 20c to the proximal end 20p are each placed at the edges of the arc shape of the central clipping arm 20c.

According to an embodiment of the present invention, the central clipping arm 20c comprises a plurality of sharp edges 20s on its sides. For example, each side of the central clipping arm 20c may comprise a plurality of circular adjacent arcs which form the sharp edges 20s at the connection points of each two adjacent arcs. Optionally or alternatively, the side arms 20a also comprise sharp edges 20as along their sides (preferably along their inner sides), e.g. a plurality of circular adjacent arcs which form the sharp edges 20as at connection points of each two adjacent arcs. These sharp edges 20s, 20as assist in providing a firm attachment of the attaching element 20 to the native cusp. Optionally or additionally, non-sharp serrations or other protrusions designs may be provided. A potential advantage of such serrations or protrusions is that they better engage the cusp and may assist in resisting blood flow, preventing device migration. Optionally or additionally, the central and/or side arms include one or more apertures or a coating or other treatment which optionally encourages tissue ingrowth and/or adhesion to the cusp.

According to an embodiment of the present invention, the sharp edges 20s, 20as arc curved (or bend) backwards or forwards (in relation to the general longitudinal axis of the attachment element 20) to engage the cusp tissue in a firmer manner. For example, the sharp edges 20s may curve (or bend) towards the outer side of the central clipping arm 20c curve to firmly engage the tissue in the belly of the cusp. Also, the sharp edges 20as may curve (or bend) towards the inner side of the frame 20m curve, to firmly engage the tissue in the cusp flow facing side. The direction of the edges may be selected to better resist pressure caused by blood flow in a matching direction.

In some embodiments of the invention, the attaching element 20 always tends to bend, thereby forming an arc, i.e., both the peripheral frame 20m (mainly the side arms 20a) and the central clipping arm 20c tend to bend to a similar extent (each of which forms an arc shape). The attaching element 20 comprises a shape memory alloy (e.g., nitinol). This potentially enables a most advantageous function. In some embodiments of the invention, attaching element 20 is manufactured by shape setting. The attaching element 20 is bent/curved (during its manufacture) and put in high temperature (as known in the art). This produces an attaching element 20 having an arc shape at room temperature, wherein the arc shape remains constant. Thereafter, if the attaching element 20 arc shape is deformed (i.e., either further bent or straitened), it will always tend to return to its arc shape state that it had in the heating process. An element of the present invention having this constant arc shape state (after being shape set to have the constant arc shape) will be referred to herein as an element having “an arc shape when undeformed”. Therefore, the attaching element 20 comprises an arc shape when undeformed. The central clipping arm 20c comprises an arc shape when undeformed, etc. It is noted that “arc shape” includes other monotonic curves as well, for example, where the radius of curvature increases or decreases along the arm. In some embodiments of the invention, a non-monotonic curve is provided and one or more arms may increase and decrease in curvature along its length. Also, it is noted that the curvature of the arms is optionally not identical between the arms.

It is noted that other materials, for example, clastic materials, such as stainless steel may be used. A potential advantage of shape memory and super clastic materials such as nitinol is that the device may be crimped or otherwise deformed during delivery to a greater extent without affecting its function after deployment.

According to a preferred embodiment, the peripheral frame 20m is substantially symmetrical. Preferably, the peripheral frame 20m comprises a longitudinal axis larger than its transverse axis (i.e. they are not equal). The central clipping arm 20c extends along the frame 20m longitudinal axis. Preferably, the frame 20m is elliptic. The frame may also comprise other shapes, e.g. oval, rectangular, etc. In some embodiments of the invention, the frame is not symmetrical (e.g., frame 20m and/or central arm 20c are not symmetrical and/or not symmetrically aligned). This may be useful if the frame is implanted in an asymmetrical part of the heart, for example, not in the plane including the mitral and aortic valves.

In some embodiments of the invention, the hollow tubular member 5 is attached to the peripheral frame 20m with the hollow tubular member proximal opening 12 near the attaching element proximal end 20p and the hollow tubular member closed distal end 7 near the attaching element distal end 20d. The distance range between the proximal opening 12 and the proximal end 20p is usually between 0.1 and 0.5 mm, e.g., preferably 0.15 mm (wherein the proximal end 20p is usually placed more proximal than the proximal opening 12). The distance range between the closed distal end 7 and the distal end 20d is usually between 0.1 and 0.5 mm (wherein the distal end 20d is usually placed more distal than the closed distal end 7). The attachment between the frame 20m and the hollow tubular member 5 may be implemented by sewing. The attachment element 20 may comprise a plurality of orifices/apertures or depressions for use of the sewing. FIGS. 2A and 2G show examples of depressions 4 at the arms 20a where the depression 4 openings face outwards. The sewing “strings” may be non absorbable surgical sutures and comprise material selected from the group consisting of nylon, polyester, PVDF and polypropylene. Their thickness is usually between 10-0 and 2-0.

In some embodiments of the invention, the curve of the attaching element 20 (both the peripheral frame 20m and the central clipping arm 20c) is curved away from the hollow tubular member 5. In other words, the hollow tubular member 5 engages the external part of the convex curve of the arc portion, i.e., the hollow tubular member 5 engages (and is attached at) the curved side which is the exterior of the arc portion.

Nevertheless, other embodiments may include the curve of the attaching element 20 (both the peripheral frame 20m and the central clipping arm 20c) curved towards the hollow tubular member 5 (engaging the internal part of the convex curve of the arc portion; i.e., the hollow tubular member 5 engages (and is attached at) the curved interior side of the arc portion), mutatis mutandis.

FIG. 2B shows a side view of the obstructing device 10. FIG. 2C shows a “top” view of the obstructing device 10, with the central clipping arm 20c curving towards the top (having an arc shape). FIG. 2D shows a side view example of the present invention obstructing device 10 about to be attached to a native cusp. Whereas FIG. 2A shows the device 10 with the frame 20m and central clipping arm 20c being in their undeformed curved arc shape, FIG. 2H shows an embodiment with the frame 20m and central clipping arm 20c being in a deformed manner-straightened.

FIG. 2D shows an option for delivery where hollow member 5 (and optionally frame 20) is mounted on a guidewire/delivery catheter, potentially allowing the cusp to be engaged or device positioned while also allowing the efficacy of member 5 in preventing regurgitation, to be estimated, by allowing member 5 to expand in blood flow. In other embodiments of the invention, member 5 is folded in a delivery capsule and the guidewire (if any) passes to the side of it, while central clipping arm 20C is positioned as shown in FIG. 2D. This may make deployment easier, but interfere with estimation of device efficacy.

The attachment element 20 may comprise connecting elements (e.g., 20q, see FIG. 2J, for example showing two such needles) connectable to the cusp. For example, the connecting elements may be biocompatible needles that slant therefrom. The connecting elements are placed on the attachment element 20 surfaces that engage the cusp, as explained herein. The biocompatible needles may pierce the cusp portions that engage the attachment element 20 and thereby enhance a firm connection of the obstructing device 10 to the cusp. Other types of connecting elements may be used, such as biocompatible pins, biocompatible spikes, etc.

According to an embodiment of the present invention, the connecting elements slant backwards or forwards (in relation to the general longitudinal axis of the attachment element 20) to engage the cusp tissue in a firmer manner. For example, the connecting elements on the central clipping arm 20c may face the outer side of the central clipping arm 20c curve to firmly engage the tissue in the belly of the cusp. Also, the connecting elements may face the inner side of the frame 20m curve, to firmly engage the tissue in the cusp flow facing side.

FIG. 2I is an image of a frame 20a including a central arm 20c of an embodiment of the attaching element 20 mounted on a cusp 21, viewed from the location of cusp cooptation towards a vessel wall. In this embodiment, both serrations (or other tissue engaging geometries) 20as, on arms 20a and serrations (or other geometries) 20s are shown. In some embodiments of the invention, such serrations are sharp enough to engage tissue but blunt enough to avoid or reduce trauma to cusp tissue, especially trauma which might cause calcification or tearing of a cusp.

For example, such serrations may be between 0. And 2 mm apart, for example, 1 mm. Optionally or additionally, such serrations are between 0.3 and 0.7 mm long, for example, 0.5 mm. a distance between arm 2c and arms 2a, while generally not constant, can be, for example, related to a cusp thickness, for example, be between 60% and 200% of a cusp thickness, for example, be between 90% and 150%, for example, be between 105% and 130%. In an example of a 0.4 mm thick cusp, the distance may be, for example, 0.5 mm on the average. In other embodiments, a distance of, for example, between 0.2 and 1.5 mm may be provided. Optionally, the distance is selected so that the cusp is not pinched hard enough to be damaged. Also, the distance may be selected so that arm 20c and arms 20a can sit in a same (curved) plane of the cusp with reduced deformation of the cusp, while still providing anchoring even under conditions of aortic outflow. This may reduce stress on the cusp and/or reduce regurgitation.

In the embodiment shown there are multiple mechanisms holding frame 20m to cusp 21, including, sideways crimp provided by the arms, serrations which both enhance the crimp (e.g., by increasing friction) and provide axial (along the frame length) anchoring and also optional needles 20q.

FIG. 2J is an image of a frame including a central arm portion of an embodiment of the attaching element 20 in a relaxed state and in a lateral view corresponding to a view along the plane of cusp 21. Also visible is a ruler (in mm), showing typical dimensions of some embodiments of the invention. Optional pins 20q are visible on a tip 20f of central arm 20c.

It is noted that the curve of arm 20c and of arms 20a is optionally not the same, with arm 20c being overcurved, e.g., to ensure snug clasping of cusp 21 therebetween. Optionally or additionally, even if the curve is the same, base 20 selectively (and elastically) aims arms 20a and arm 20c in different directions. Optionally or additionally, it is noted that the baseline stiffness of arms 20a can be set to be greater than that of arm 20c, e.g., due to their greater combined cross-section, which causes arm 20c to bend more in response to deployment.

It is also noted that when engaging a cusp 21, cusp 21 would be above arms 20a as shown in FIG. 2J with arm 20c above cusp 21 and needles 20q pointing down into the cusp.

The present invention, in some embodiments thereof, provides a device that closes the malcoaptation gap by filling it with an artificial cusp that self-fits the gap when filled with blood and/or causes the cusps to adapt to the device geometry. A potential advantage of some embodiments of the present invention is providing a better tolerance to the implantation position than in the prior art leaflet prolongation method (in US 2015/0230919), and a more efficient closure of the gap, both or either of which may be provided by a potentially more stable anchoring of the instant design, which may prevent movement axially and/or radially along the cusp, especially movement within a cardiac cycle and/or movement between cardiac cycles. Furthermore, a second device can be implanted next to the first one if needed. Furthermore, the method of attachment to the native cusp can be more efficient and less complicated than that of the method of WO 2020026234 (e.g., by potentially avoiding string manipulation during insertion and/or not using connecting elements biocompatible pins).

The present invention, in some embodiments thereof, relates to a method for delivering an obstructing device (e.g. the obstructing device as defined herein) via a delivery system to the intended heart valve location, deploying and correctly positioning the obstructing device within the heart valve intended location such that it begins to function by improving the function of the heart valve. The present invention attachment element 20 curving feature is optionally utilized to attach the obstructing device 10 to a respective native cusp in. The obstructing device 10 is delivered through the delivery system where its attachment to a respective native cusp is carried out.

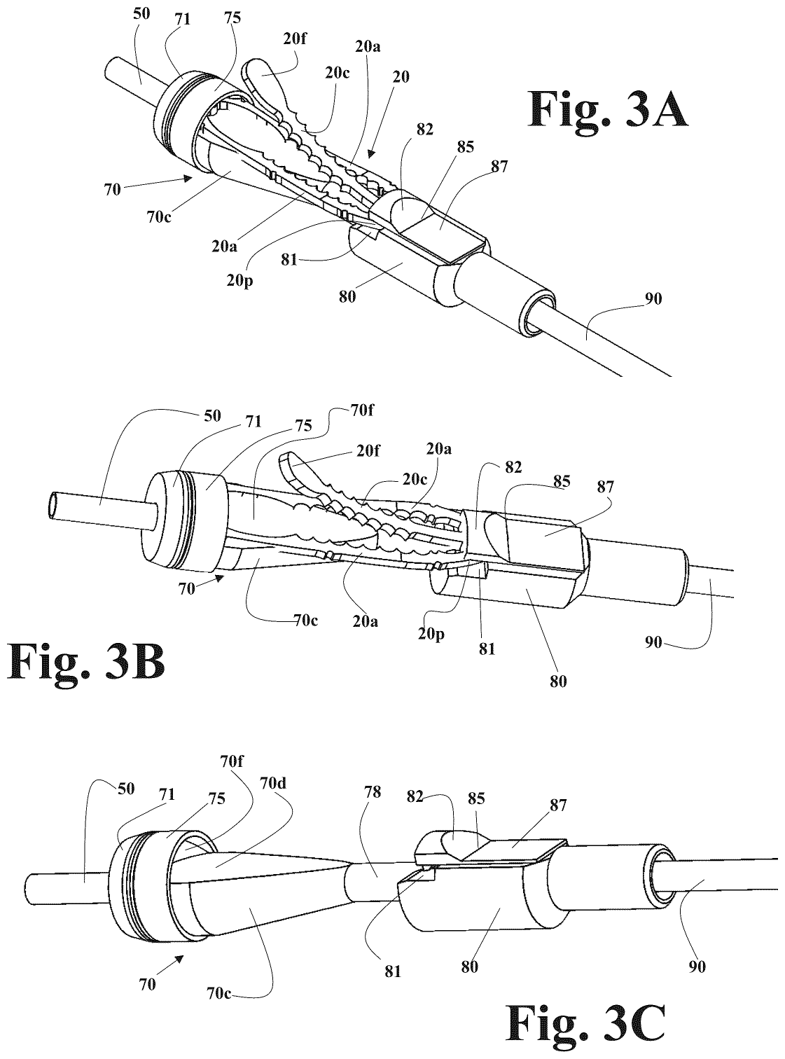

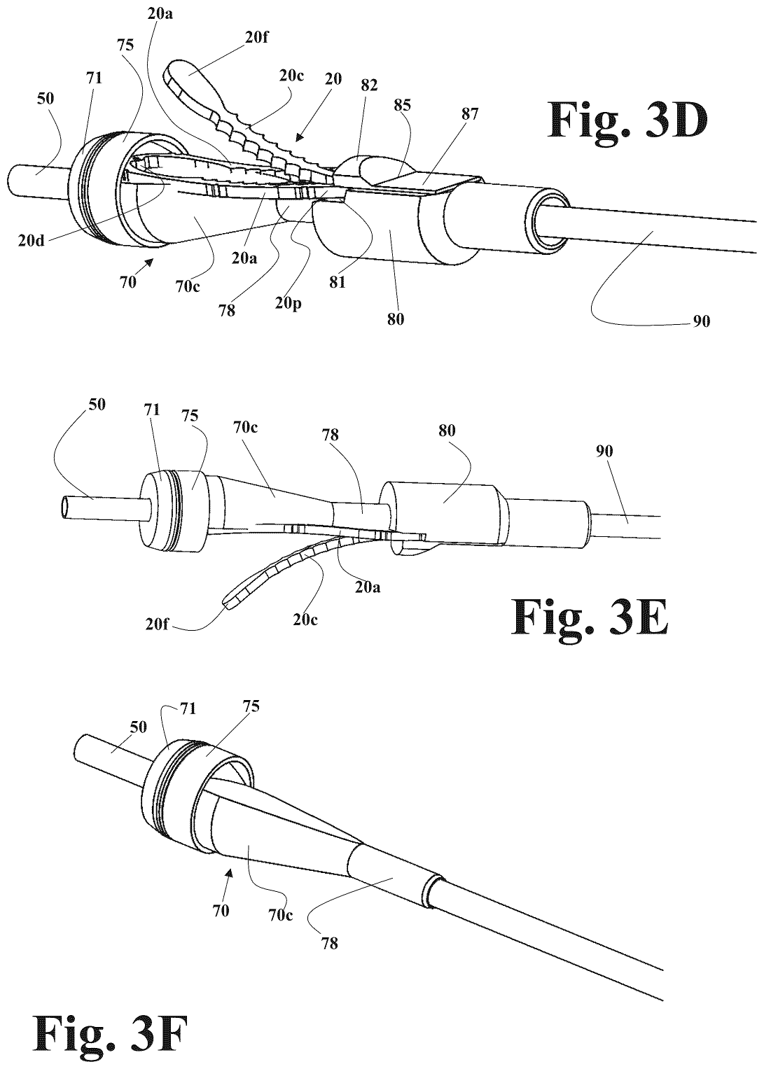

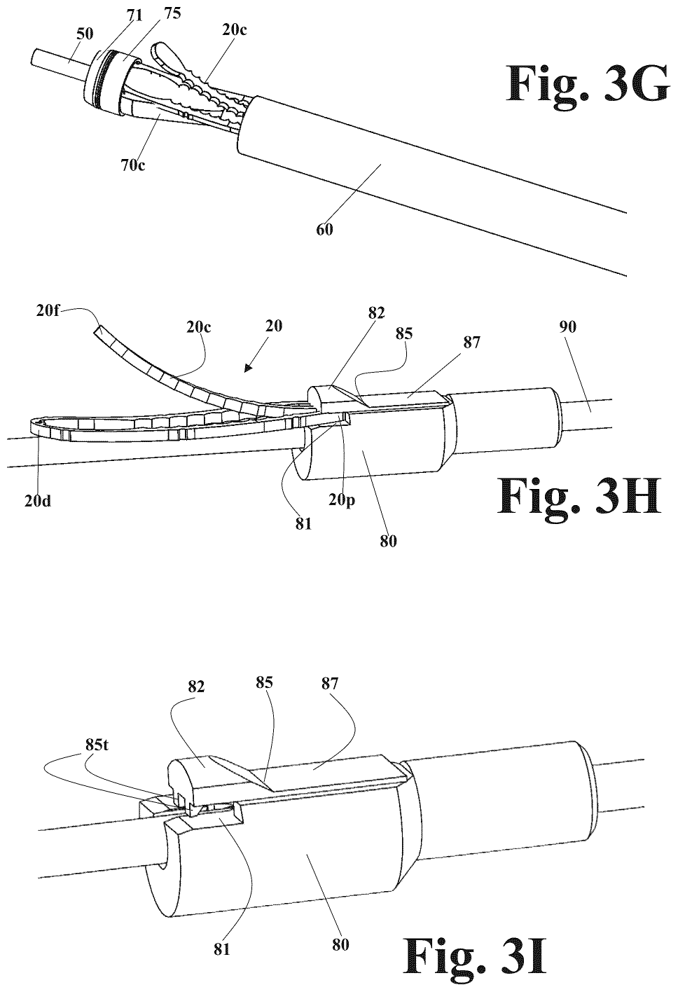

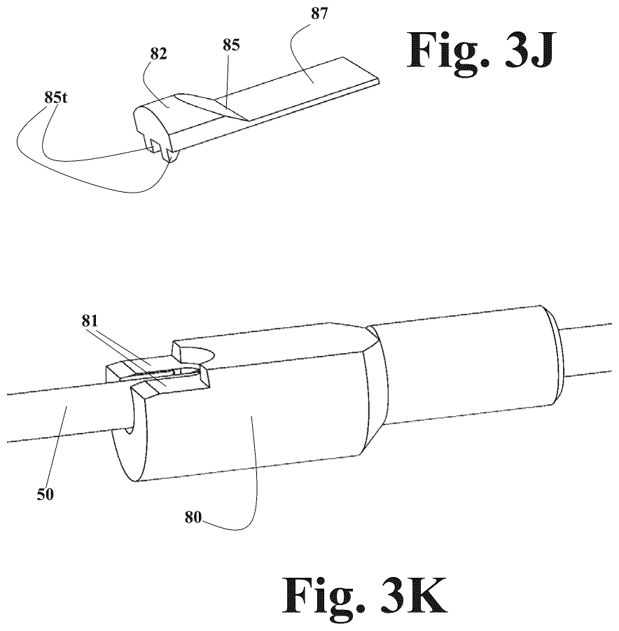

The present invention delivery system, in some embodiments thereof, comprises movable elements (e.g., as will be explained herein) in order to safely hold, control and mount the present invention device on a native cusp. FIGS. 3A-3K show various portions of the present invention delivery system, according to some embodiments. The delivery system comprises a guide wire (not shown in FIGS. 3A-3K) and a wire lumen 50 (e.g. catheter) configured to pass over the guide wire, for example, in an “over the wire delivery” manner. The guide wire may optionally be an extra stiff guide wire. For example, the guide wire is of type 035 inch. The delivery system further comprises an outer sheath 60 a distal holder 70 (e.g., in the form of distal cone 70) and a proximal holder 80. The obstructing device 10 is configured to rest on and be retained by the distal cone 70 and proximal holder 80. The attaching element 20 of the obstructing device 10, without the tubular member 5, is shown in most of these figures for the purpose of simplicity. However, it would be understood that the present invention system and method would include the entire obstructing device 10 with the tubular member 5 in a crimped/folded/compressed/collapsed configuration (e.g. a pre-load state) attached to the attaching element 20 for example as explained herein.

The delivery system comprises a proximal lumen 90 (e.g. as part of a catheter) fixedly connected to the proximal holder 80. The proximal lumen remains stationary during one or more of the method stages, as will be explained herein. The distal end of proximal lumen 90 (not shown) terminates within the proximal holder 80. The wire lumen 50 passes through the proximal lumen 90. The proximal holder 80 comprises an inner bore which the proximal lumen 90 passes through. Preferably, the proximal lumen 90 is fixedly connected to a portion of the inner bore and the wire lumen 50 continues distally (from within the distal end of the proximal lumen 90) along the inner bore to the distal cone 70. The wire lumen 50 passes through a second bore within the distal cone 70. The wire lumen 50 is fixedly attached to the distal cone 70 (typically to a portion of the distal cone 70 inner bore).

The delivery system optionally comprises a proximal handle (not shown) for controlling the delivery system (e.g. pushing/pulling the handle forwards/retracts the wire lumen 50 and thereby the holders sheath and proximal lumen 90). The handle comprises appropriate knobs (as known in the art) to control the movements of the wire lumen 50, the proximal lumen 90 and the outer sheath 60. The movement control of these elements potentially enable for a most efficient retaining of the obstructing device 10 and its gradually deployment as will be explained.

FIG. 3A shows the distal cone 70 and proximal holder 80 retaining the attaching element 20. The distal cone 70 and proximal holder 80 are distanced from one another such that the attaching element 20 is tightly retained (the distal cone 70 retaining distal end 20d and the proximal holder 80 retaining proximal end 20p.

The distal cone 70 comprises a distal front member 71. The distal front member 71 may be in the form of a disc or in the form of a “front cone” tapering distally with a proximal circular disc shape. The wire lumen 50 passes through the distal front member 71 (preferably via its center). The distal cone 70 (according to this embodiment) comprises a “sliced” cone shaped element 70c attached at its distal end to the distal front member 71. The cone shaped element 70c has a flat surface 70f. It could be considered that the cone shaped element 70c has a bottom partial cone shape tapering proximally and has a top flat surface 70f. The distal cone 70 comprises a ring-shaped element 75 extending proximally from the distal front member 71. Optionally, the distal portion of flat surface 70f has a diagonal flat surface 70d that may assist in retaining the distal portion of attaching element 20. The distal cone 70 comprises a proximal spacer 78 (preferably tubular). The proximal side of the spacer 78 is engageable with the distal end of proximal holder 80. The spacer 78 length is such that when it engages the distal end of proximal holder 80 the attaching element 20 is secured in a good manner. The wire lumen 50 passes through the spacer 78. In other embodiments the distal holder can have other shapes than the distal cone 70 (e.g. tubular), but still may comprise one or more of a top flat surface, a distal front member, a ring, spacer, inner bore, being fixedly connected to wire lumen 50, etc., mutatis mutandis.

FIG. 3B shows another angle of FIG. 3A. FIG. 3C shows the elements of FIGS. 3A and 3B without the attaching element 20. FIG. 3D shows the elements of FIGS. 3A and 3B in a position where the interior of ring 75 and the diagonal flat surface 70d may be seen. FIG. 3E shows the elements of FIGS. 3A and 3B in an “upside-down” manner. FIG. 3F shows the distal cone 70 and the wire lumen 50 only. FIG. 3G shows the outer sheath 60 partially covering the attaching element 20. FIG. 3H shows the proximal holder 80 retaining the attaching element 20 (without showing the distal cone 70).

The proximal holder 80 comprises a distal surface 81 and a lock spring element 85, such that the attaching element 20 proximal end 20p is retained between the distal surface 81 and an optional lock spring element 85. The lock spring element 85 comprises a distal portion 82 such that at least a distal portion of distal portion 82 is placed above a proximal portion of the distal surface 81. Thus, a gap is formed between a portion of the distal portion 82 and the distal surface 81, wherein the attaching element 20 proximal end 20p is retained within said gap. The lock spring element 85 further comprises a proximal portion 87.

The lock spring element 85 comprises a spring mechanism (not shown), for example in the form of an elastic tab (or other spring shape, such as a loop) that pushes the distal portion 82 of the lock spring element 85 away from the central axis of the proximal holder 80 (e.g., when not retained by the sheath 60 as will be explained). The central axis of the proximal holder 80 is the center of its inner bore (along the proximal-distal direction). For example, the spring mechanism may comprise a spring anchored between a portion of the proximal holder 80 and a portion of the lock spring proximal portion 87. The distal surface 81 may be split into two portions by the proximal holder 80 bore, partially “overlapping” from beneath (as can be seen in FIG. 3K).

FIG. 3I shows the proximal holder 80, the wire lumen 50 and the proximal lumen 90. FIG. 3K shows the elements of FIG. 3I without the lock spring element 85. FIG. 3J shows the lock spring element 85. The lock spring element 85 preferably comprises two teeth elements 85t (protrusions) extending downwards from distal portion 82 of the lock spring element 85, for assisting in retaining the attaching element 20 proximal end 20p. When retaining the attaching element 20, the teeth elements 85t are placed preferably such that each of the teeth elements 85t is positioned in a respective open space between the central clipping arm 20c and a respective side arm 20a.

The delivery of the obstructing device 10 is carried out as follows. First the attaching element 20 is positioned such that it rests on the distal cone 70 top flat surface 70f and on the distal surface 81 of the proximal holder 80. The attaching element 20 is retained at its distal end 20d by the inner surface of the ring-shaped element 75 which engages distal end 20d from above and/or from the side. The diagonal flat surface 70d may also contribute to its tight fixation. The attaching element 20 is retained at its proximal end 20p by the lock spring element 85 distal portion 82 and teeth 85t. The outer sheath 60 pushes the distal portion 82 towards the proximal holder 80 central axis thereby retaining the proximal end 20p.

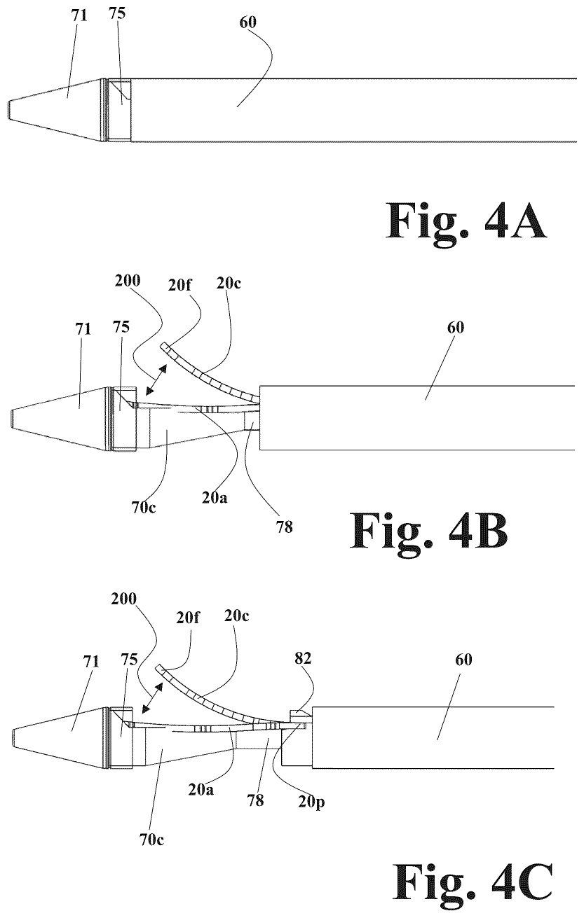

FIG. 4A shows the initial configuration with the outer sheath 60 fully covering the obstructing device 10. The distal end of the outer sheath 60 engages the proximal end of ring 75. In this configuration the distal front member 71 is in the shape of a front cone tapering distally with the wire lumen 50 passing therethrough. In other embodiments the wire lumen 50 terminates at a distal section of the distal holder 70 (e.g. at the most distal portion of the distal front member 71), wherein the guidewire continues distally out of the distal holder 70.

In this figure, the attachment element 20 frame 20m is initially placed in a flat position and the central clipping arm 20c (not shown) is not fully curved, only curved until engaging the outer sheath 60. The obstructing device 10 is advanced to the target location during which the outer sheath 60 continuously covers it and engages ring 75. The distal cone 70 proximal spacer 78 engages with the distal end of proximal holder 80 during this advancement, thereby securing the obstructing device 10.

According to a preferred embodiment, the delivery system is such that the handle may advance the wire lumen 50 and thereby the distal and proximal holders and outer sheath 60 connected thereon which move together when wire lumen 50 is advanced/retracted. The handle comprises abilities to move the outer sheath 60 and/or the proximal sheath 90 distally or proximally in relation to the wire lumen 50, as known in the art. This enables the gradual deployment of the obstructing device 10.

When arriving at the target location, the sheath 60 is proximally retracted (by the handle), gradually unsheathing the obstructing device 10. FIG. 4B shows the sheath 60 partially retracted. Part of the ring 75 is shown in a transparent form in FIGS. 4B-4C for a better understanding. When the sheath proximally passes the central clipping arm 20c distal portion, the central clipping arm 20c curves and a gap 200 is formed between the central clipping arm 20c and the frame 20m surface (the common surface containing the side arms 20a). The distal free end 20f extends beyond the imaginary line where the sheath 60 has restrained it up until now.

With the optional assistance of fluoroscopy and/or ultrasound imaging the attaching element 20 (with arm 20c defining a gap 200 from arms 20a) is moved relative to the native cusp so the native cusp is placed within the gap 200 such that the native cusp is positioned between the central clipping arm 20c and the frame 20m. One manner for such placement is to advance the gapped device 10 forward when the cusp is not in contact with other cusps along is free edge and thereby capture the cusp in the gap. The attaching element 20 is positioned such that the central clipping arm 20c is placed engageable with the belly of the cusp, while the peripheral frame 20m is engageable with the cusp flow facing side. Other delivery embodiments may include a two-way steering mechanism.

In some embodiments of the invention, rapid pacing or other cardiac immobilization methods are used to reduce the movement of the cusp. Failure to capture is optionally remedied by retracting element 10 and advancing it again. Rapid pacing is optionally stopped once the cusp is in the gap, optionally, before the gap is closed.

Thereafter, the wire lumen 50 is moved distally by the handle and the proximal lumen 90 is maintained in place (by the handle). This causes the distal cone 70 to move distally while the attachment element 20 is retained at its proximal end 20p by teeth 85t. The distal end 20d is released from the ring 75 and surface 70f retainment and curves accordingly with the central clipping arm 20c thereby closing the gap 200 and thereby “clipping” onto the native cusp and being firmly mounted thereon (wherein the central clipping arm 20c tightly engages the belly of the cusp and the peripheral frame 20m tightly engages the cusp flow facing side).

Thereafter, the sheath 60 is further retracted proximally beyond the distal portion 82 of the lock spring element 85 thereby releasing the lock spring element 85 pushing it away from the central axis of proximal holder 80 and thereby the teeth elements 85t move away from the central axis of proximal holder 80 and release the attachment element 20 proximal end 20p. This lock spring safety feature is aimed to prevent early implant release prior to optimal positioning on the selected cusp. After its release, the obstructing device is fully deployed and mounted on the native cusp where the tubular member 5 is positioned in the regurgitant orifice (the malcoaptation gap) when the heart valve is in a closed state thus preventing blood leakage. The delivery system is thereafter retracted.

In some embodiments of the invention, even after frame 20m is closed, as long as base 20p is being help, element 20 may be pulled off the cusp by retraction this may cause some damage to the cusp.

An embodiment of the present invention delivery method will be explained in relation to the aortic valve, but may similarly be delivered to the pulmonary valve (and with many aspects to the mitral and tricuspid valves) cusps/leaflets, mutatis mutandis.

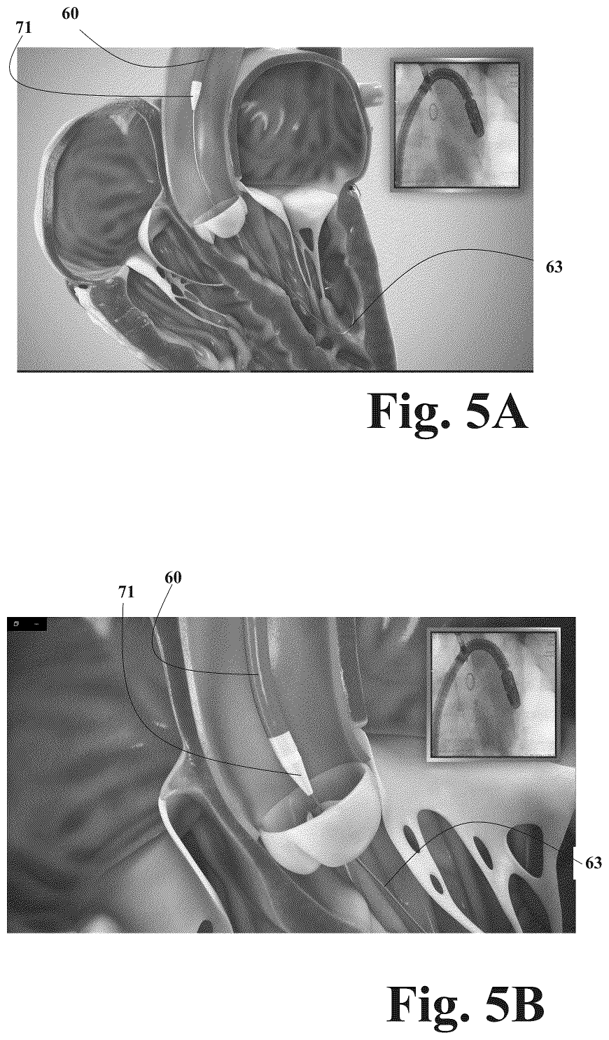

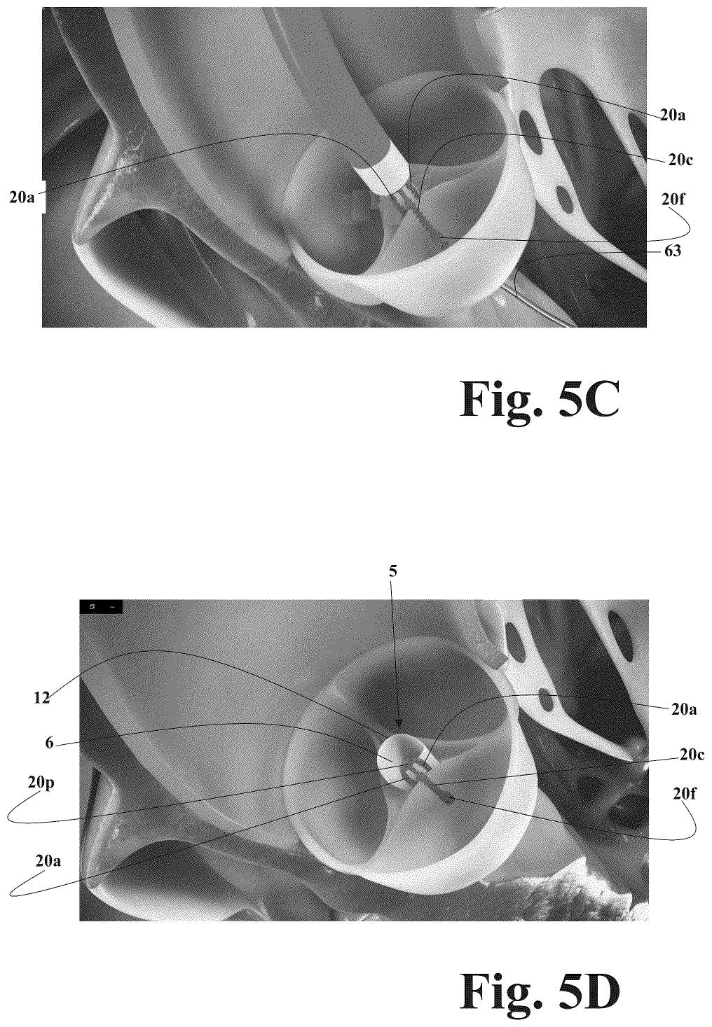

The present invention, in some embodiments thereof, relates to a method for implanting the obstructing device as explained herein. The method is partially shown in FIGS. 5A-5D. Portions of the method are also taught in the description relating to the present invention device and system as explained herein. The method comprises the following steps:
creating an opening in a blood vessel;
inserting an introducer sheath;
inserting a guide wire 63 through the introducer sheath and passing it through the blood vessel all the way to the heart valve and therethrough to the respective heart chamber; other manners of providing access to the valve may be used as well.
providing a delivery catheter system, for example as explained herein, for example, allowing selective release of the central arm and moving device 10 with the central arm selectively released and then releasing the frame. One example comprises:
   The obstructing device as explained herein;
   a distal holder 70 retaining the peripheral frame distal end 20d;
   a proximal holder 90 retaining the peripheral frame proximal end 20p;
   a wire lumen 50 connected to a handle and distally and proximally movable by said handle, and fixedly attached to said distal holder 70;
   a proximal lumen 90 connected to said handle and fixedly attached to said proximal holder;
   wherein said wire lumen 50 passes through said proximal lumen 90;
   an outer sheath 60 distally and proximally movable by said handle, configured to cover the proximal holder 80 and part of the distal holder 70, wherein the central clipping arm 20c is deformed by being at least partially straightened by the outer sheath 60 restricting it;
passing the delivery catheter system distally (FIG. 5A), by passing the wire lumen 50 over the guidewire 63 until the distal portion of the outer sheath 60 arrives at the ascending aorta (e.g. approximately 5 mm above the aortic valve) as shown in FIG. 5B; Optionally, the positioning of the guidewire helps align the device with the cusps in a radial direction with respect to the aorta. retrieving the outer sheath 60 proximally (unsheathing the obstructing device 10 thereby beginning to release the obstructing device 10, wherein during this stage the proximal lumen 90 and wire lumen 50 remain stationary while the outer sheath 60 moves proximally) to an extent where the central clipping arm 20c curves to its arc shape when undeformed;
positioning the obstructing device 10 in the correct intended position to be attached to the native cusp or leaflet (this positioning comprises rotating the obstructing device 10 such that the native cusp is positioned between the central clipping arm 20c and the frame 20m, and forwarding the obstructing device distally to the desired extent for attachment);
forwarding the wire lumen 50 distally while the proximal lumen 90 remains stationary (by means of the handle), thereby releasing the peripheral frame distal end 20d from being retained by the distal holder 70, and thereby having the frame 20m curve to its arc shape when undeformed (and thereby the central clipping arm 20c is placed tightly engaging the belly of the cusp, while the peripheral frame 20m tightly engages the cusp flow facing side);
retrieving the outer sheath 60 proximally to an extent where the peripheral frame proximal end 20p is released from being retained by the proximal holder 80 (thereby releasing the obstructing device 10 to be completely deployed); the procedure may now be completed and the delivery system retrieved, for example by:
proximally retrieving the outer sheath 60, the distal holder 70 and proximal holder 80;
proximally retrieving the guide wire 63;
removing the introducer sheath;
closing the blood vessel.

Preferably, the retrieving of the outer sheath proximally to an extent where the peripheral frame proximal end 20p is released from being retained by the proximal holder 80, comprises retrieving the outer sheath to an extent such that a lock spring element is released thereby releasing the peripheral frame proximal end 20p from being retained by the proximal holder 80.

An expanded aspect of the method (which may be understood in conjunction with the method description hereinabove) is described below. The method comprises:
Making a skin incision (e.g. in the groin).
Creating an opening in a blood vessel (e.g. the femoral artery) and inserting an introducer sheath (e.g. a 6Fr introducer sheath).
Optionally inserting a closure device.
Inserting a stiff guide wire 63 (e.g. a stiff 0.35′ wire) through the introducer sheath and passing it through the femoral artery.
Optionally replacing the 6Fr introducer sheath with a large 11 to 16Fr introducer sheath.
Optionally replacing the stiff guide wire with a regular guide wire and advancing it all the way up to the aorta and through the opening in the aortic valve and into the left ventricle. Preferably, the guide wire is placed such that it contours to the inner cavity of the left ventricle all the way to the apex, preferably using a pigtail catheter. In some embodiments the first guide wire is the only guide wire inserted (advancing it all the way up to the aorta and through the opening in the aortic valve and into the left ventricle, etc.) for the whole delivery procedure.
It should be noted that the insertions of the guide wire(s) explained herein may be according to the basilica method.
Optionally, replacing the regular (soft) guide wire with a stiff guide wire 63. Such replacement may assist in supporting the device maneuvering acts. A different appropriate guidewire may be used.
Passing a delivery catheter system distally (FIG. 5A), the delivery system is as explained herein, preferably comprising:
   a distal holder 70 comprising:
      a distal front member 71;
      a ring-shaped element 75 extending proximally from said distal front member 71;
      a top (preferably) flat surface 70t;
   a proximal holder 80 comprising:
      a distal surface 81;
      a lock spring element 85 comprising a spring mechanism configured to push the distal portion 82 of said lock spring element 85 away from the central axis of the proximal holder 80, and
      wherein a gap is formed between the distal portion 82 of said lock spring element 85 and a portion of said distal surface 81;
   a wire lumen 50 connected to a handle and distally and proximally movable by said handle;
   a proximal lumen 90 connected to said handle;
   an outer sheath 60 distally and proximally movable by said handle, configured to cover the proximal holder 80 and part of the distal holder 70 and engageable with the distal holder ring-shaped element 75 (such that when engaged, the engagement forms a closed/sealed closure);
   wherein said wire lumen 50 passes through said proximal lumen 90;
   wherein said wire lumen 50 is fixedly connected to said distal holder 70; and
   wherein said proximal lumen 90 is fixedly connected to said proximal holder 80;
   the obstructing device 10 as explained herein, wherein the peripheral frame distal end 20d is placed within the ring-shaped element 75; and
   wherein the peripheral frame proximal end 20p is placed within the gap.

In this system, the guide wire may pass through the wire lumen 50. The wire lumen 50 can pass in an “over the wire delivery” manner over wire 63 (while wire 63 passes through wire lumen 50).
When the distal portion of the inner sheath arrives at the ascending aorta (e.g. approximately 5 mm above the aortic valve) as shown in FIG. 5B, retrieving the outer sheath 60 proximally (unsheathing the obstructing device 10) thus beginning to release the obstructing device 10. During this stage the proximal lumen 90 and wire lumen 50 remain stationary while the outer sheath 60 moves proximally. The outer sheath 60 is proximally retrieved to an extent where the central clipping arm 20c curves (but still covering the lock spring element 85 distal portion 82).
The next step comprises positioning the present invention obstructing device 10 in the correct intended position prior to attaching it to the cusp. This positioning comprises rotating the obstructing device 10 such that the native cusp is positioned between the central clipping arm 20c and the frame 20m (the central clipping arm 20c is placed engageable with the belly of the cusp, while the peripheral frame 20m is engageable with the cusp flow facing side. This may be carried out based on imaging modality (e.g. a fluoroscopy and ultrasound imaging) by manipulating the delivery system such as clock or counter-clock rotation. This is carried out by rotating the wire lumen 50 (by means of the system handle) which in turn rotates the distal cone 70 and proximal holder 80 and thus the device 10 which is retained thereon them. The wire 63 may be pushed or pulled to maneuver the catheter as known in the art for correct placement of the obstructing device 10.
Once at the required rotated position (e.g. to engage the predetermined aortic cusp) the distal cone 70 and proximal holder 80 are pushed forward (distally) by the wire lumen 50 advanced distally (along with the proximal lumen 90) such that the native aortic cusp is within the gap 200 (between the central clipping arm 20c and the frame 20m) as shown in FIG. 5C.
Then, the wire lumen 50 is moved distally and the proximal lumen 90 remains stationary (by means of the handle), thereby the front end of the attachment element 20 is released from the distal cone 70 ring 75 and curves accordingly with the central clipping arm 20c. Thereby, the central clipping arm 20c is placed tightly engaging the belly of the cusp, while the peripheral frame 20m tightly engages the cusp flow facing side.
Once position on the cusp is verified by the imaging modalities, the outer sheath is proximally retrieved to an extent proximal to the lock spring element 85 distal portion 82, thereby causing the lock spring element 85 to move away from the proximal holder 80 central axis, thereby releasing the obstructing device 10 to be completely deployed (the attachment element 20 forms a “clip” on the native cusp (locking it in place) with the hollow tubular member 5 attached to the frame 20m and being placed at the regurgitant orifice (the malcoaptation gap) when the native valve closes).
Thereafter, the outer sheath 60, the distal cone 70 and proximal holder 80 are proximally retrieved by the handle in an “over the wire” manner.
The guide wire 63 is then proximally retrieved. The obstructing device 10 is mounted on the native cusp and working (FIG. 5D). While some gaps are shown, this need not be the case. Even if some gaps remain, regurgitation is expected to be substantially reduced (e.g., between 50% and 70%, between 70% and 90% or more) by obstructing device 10.
Then the introducer sheath is removed and the artery is closed.

The obstructing device implant can be withdrawn back into the delivery system prior to full deployment, when readjustment for optimal positioning or a bailout is required. At any stage before the final deployment (by the lock spring element 85) there is an option to reposition or retrieve the obstructing device 10, allowing the personnel cardiologist multiple attempts at effectively reducing regurgitation with feedback from the imaging modalities. For example, before the final deployment step (retracting outer sheath 60 and releasing the lock spring 85) the obstructing device 10 can still be repositioned/retrieved by re-sheathing the outer sheath 60 (i.e., advancing it distally), etc.

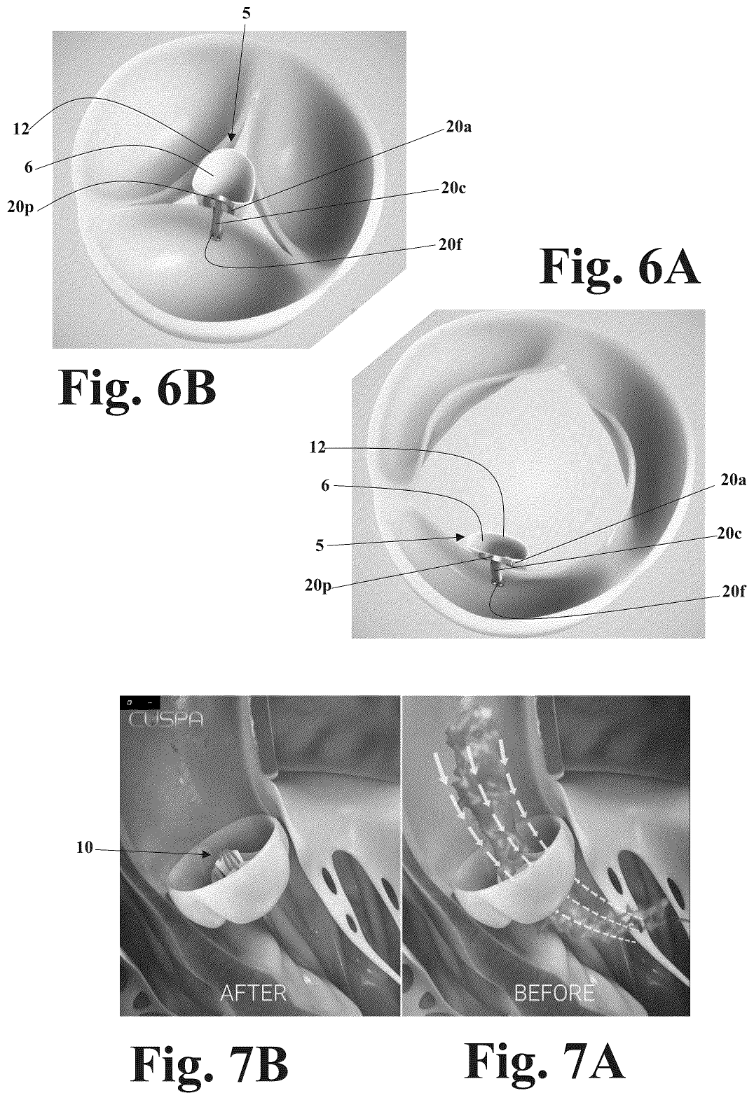

FIG. 6A shows the device functioning in a valve open state and FIG. 6B shows the device functioning in a valve closed state preventing blood leakage into the left ventricle. The remaining gaps shown in this computer generated image would typically be closed, at least in most part in the body due to the compliance of the cusps. Some amount of regurgitation may remain. Optionally, regurgitation is reduced by at least 50%, 70%, 80% or more or intermediate amounts. FIG. 7A shows the heart valve before implanting the present invention obstructing device. It can be shown that due to the blood leakage, a considerable amount of blood flows back through the valve. FIG. 7B shows the present invention obstructing device 10 preventing blood leakage.

According to another aspect of the present invention, e.g., for delivery to the mitral and tricuspid valves, the obstructing device 10 is mounted in the opposite direction within the delivery system i.e. with the hollow tubular member opened portion distally and the hollow tubular member closed portion proximally. According to this aspect of the present invention the terms “distal” and “proximal” defining the elements of the obstructing device 10 are switched, e.g. the opening (12) will be referred to as the distal opening, the distal/proximal portions of the attachment element are switched, etc. mutatis mutandis.

According to this aspect of the present invention the delivery system and method of insertion are almost the same, and the following portion will mainly emphasize on the differences. The attachment element according to this embodiment will be referenced 120.

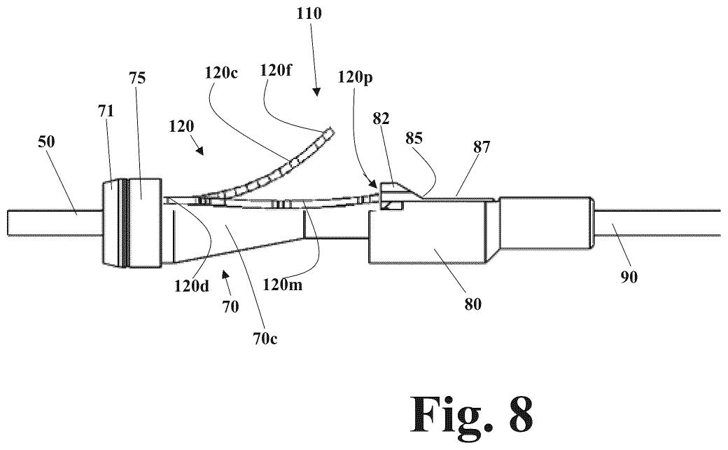

Reference is made to FIG. 8 that shows the attachment element 120 placed in the opposite direction than that of FIGS. 3A-3K and FIGS. 4A-4C. In this embodiment the central clipping arm 120c extends proximally from the frame 120m distal side. Therefore, during delivery, the outer sheath 60 moves proximally until it passes the proximal free end 120f and the central clipping arm 120c curves to an arc shape.

According to one embodiment of the present invention, the main differences between the method for delivering the obstructing device to the mitral or tricuspid cusps/leaflets and the method for delivery to the aortic and pulmonary cusps/leaflets comprise:
Providing an obstructing device with the central clipping arm 120c extending proximally from the frame 120m distal side 120d (and not distally from the frame 120m proximal side 120p). The device itself may be larger than for the aorta—the frame and/or expanding member. The access is optionally through the atria which may assist in avoiding damage to the chordea. Access from the ventricle is possible as well, in which case the device orientation during delivery may be similar to that of an aortic device.
Passing the delivery catheter system distally, by passing the wire lumen 50 over the guidewire 63 until the distal portion of the outer sheath 60 passes the respective tricuspid or mitral valve (and not stopping before arriving at the aorta or pulmonary valve).
retrieving the outer sheath 60 until it completely passes the central clipping arm 120c.
The positioning of the obstructing device 110 in the correct intended position to be attached to the native cusp or leaflet mainly comprises retrieving proximally (and not forwarding distally) the obstructing device to the desired extent for attachment.

This aspect of the present invention method comprises:
creating an opening in a blood vessel;
inserting an introducer sheath;
inserting a guide wire through the introducer sheath and passing it through the blood vessel all the way to the heart valve and therethrough to the respective heart chamber;
providing a delivery system comprising:
   an obstructing device comprising:
   a hollow tubular member comprising:
      a distal opening at its distal end;
      a substantially tubular surface extending proximally from said distal opening;
      a proximal end, being either closed or comprising a small orifice;
   an attachment element comprising:
      a peripheral frame having a distal end, a proximal end and two side arms, each of said side arms being attached to said distal end and proximal end;
      a central clipping arm extending proximally from said distal end and having a free end;
   wherein said hollow tubular member is attached to said peripheral frame; and
   wherein said peripheral frame and central clipping arm are both curved and have an arc shape when undeformed;
a distal holder retaining the peripheral frame distal end;
a proximal holder retaining the peripheral frame proximal end;
a wire lumen connected to a handle and distally and proximally movable by said handle, and fixedly attached to said distal holder;
a proximal lumen connected to said handle and fixedly attached to said proximal holder;
wherein said wire lumen passes through said proximal lumen;
an outer sheath distally and proximally movable by said handle, configured to cover the proximal holder and part of the distal holder, wherein the central clipping arm is deformed by being at least partially straightened by the outer sheath restricting it;
passing the delivery catheter system distally by passing the wire lumen over the guidewire until passing the respective heart valve cusp or leaflet;
retrieving the outer sheath proximally to an extent where the central clipping arm curves to its arc shape when undeformed;
positioning the obstructing device in the correct intended position to be attached to the native cusp or leaflet (including pulling proximally to mount on the native cusp);
forwarding the wire lumen distally while the proximal lumen remains stationary thereby releasing the peripheral frame distal end from being retained by the distal holder, and thereby having the peripheral frame curve to its arc shape when undeformed and thereby having the central clipping arm placed tightly engaging the belly of the cusp while the peripheral frame tightly engages the cusp flow facing side;
retrieving the outer sheath proximally to an extent where the peripheral frame proximal end is released from being retained by the proximal holder, thereby releasing the obstructing device to be completely deployed;
proximally retrieving the outer sheath, the distal holder and proximal holder;
proximally retrieving the guide wire;
removing the introducer sheath;
closing the blood vessel.

The attachment element (both the side arms 20a and the central clipping arm 20c) tend to curve forming an arc of a circle with a radius angle usually between 10 and 20 degrees.

The total length of the frame 20m is preferably between 15 and 25 mm. The total width of the frame 20m is preferably between 3 mm and 5 mm. The thickness of the frame 20m (any one of its portions) is preferably between 0.1 mm and 1 mm. The width of each of the arms 20a is preferably between 0.2 and 0.8 mm.

The length of the central clipping arm 20c is preferably between 5 and 15 mm. Its width is preferably between 0.2 and 1.5 mm. Its thickness is preferably between 0.1 and 0.8 mm.

The general length of the tapering tubular member 5 is usually between 10 and 25 mm. The diameter of the proximal opening 12 is usually between 4 and 8 mm. FIG. 2D shows an example of the proximal opening 12 having a 6 mm diameter and the distal end having a 2 mm diameter.

The diameter of the tubular member 5 frame wires (e.g. elements 12, 13, 14, 15, 16) is usually between 0.1 and 1 mm. The thickness of the membrane of the tubular member 5 is usually between 0.1 and 1 mm.

The delivery system wire lumen 50 has a diameter preferably between 0.5 and 2.5 mm. The delivery system proximal lumen 90 has a diameter preferably between 1 and 4 mm.

The proximal holder 80 has a general diameter preferably between 1 and 4 mm. Its length is preferably between 10 and 50 mm.

The lock spring element 85 distal portion 82 has a height preferably between 0.1 and 3 mm. The length of the proximal holder 80 distal surface 81 is preferably between 3 and 20 mm.

The length of each tooth 85t is usually between 0.1 and 0.8 mm. Its width is usually between 0.1 and 0.8 mm. Its thickness (height) is usually between 0.1 and 0.8 mm.

The distal cone 70 has a length preferably between 10 and 50 mm. The distal front member 71 has a diameter preferably between 2 and 7 mm.

The ring 75 has a diameter preferably between 2 and 7 mm. Its length (along the delivery system longitudinal axis) is preferably between 1 and 20 mm. Its thickness is preferably between 0.2 and 1.5 mm.

The attachment element 20, frame wires, the connecting elements may comprise material selected from the group consisting of metal alloy and shape memory alloy. The attachment element 20, frame wires, the connecting elements may comprise material selected from the group consisting of nitinol, stainless steel, and cobalt chromium.

The lumens 50, 90, and the outer sheath 60 may be made of a material selected from the group consisting of a metal alloy and Pebax. Preferably, each of these lumens comprises several layers of polymer materials.

According to an embodiment of the present invention, the proximal holder 80 and distal holder 70 may comprise stainless steel and/or durable polymers.

The distal front member 71 may comprise a soft tip (comprising soft material). The delivery system outer sheath 60 comprises an outer diameter preferably between 12 and 18 Fr. (e.g., preferably 14 Fr.). The outer diameter of sheath 60 and soft tip of distal front member 71 may allow for low friction advancement through the main vessels, minimizing the risk of damage to the vessels.

### Example

In a proof of concept study on 3 pigs Aortic regurgitation (AR) was induced by pulling the aortic root, and then the present invention obstructing device was implanted followed by re-induction of AR. Transcatheter implantation procedure of the device was developed based on CT planning and demonstrated in healthy pigs. The results—In all 3 pigs moderate-severe AR was successfully induced by pulling on the aortic root. Following the obstructing device implantation induction of AR was reduced to none or trivial (e.g., reduced by 80% or more) based on epicardial echocardiography doppler. Transcatheter implantation of the obstructing device (e.g., using a design similar to that shown in FIGS. 21-2J, but with an expanding member) in healthy pigs was repeatedly demonstrated using a dedicated delivery system.

It is expected that during the life of a patent maturing from this application many relevant frames will be developed; the scope of the term frames is intended to include all such new technologies a priori.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

While some of the embodiments of the invention have been described by way of illustration, it will be apparent that the invention can be carried into practice with many modifications, variations and adaptations, and with the use of numerous equivalents or alternative solutions that are within the scope of a person skilled in the art, without departing from the spirit of the invention, or the scope of the claims.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An obstructing device mountable on a heart valve cusp or leaflet, in a valve lumen having a flow axis between an upstream direction and a downstream direction, comprising:
a hollow tubular member, comprising:
an attachment element configured to trap said leaflet or cusp comprising:
a peripheral frame having a distal end, a proximal end, and two side arms;
a central clipping arm extending distally from said proximal end, located between said side arms and having a free end, so that said cusp or leaflet can be trapped between said frame and said clipping arm;
wherein said hollow tubular member is attached to said peripheral frame;
wherein said peripheral frame and said central arm are each elastically deformable and have a preferred bending plane to apply clamping force towards said leaflet or cusp when said leaflet or cusp is trapped therebetween; and
wherein said peripheral frame and said central arm are each curved in their respective preferred bending plane.

2. An obstructing device according to claim 1, wherein said peripheral frame and said central arm are curved in an arc shape in a resting state thereof.

3. An obstructing device according to claim 2, wherein the hollow tubular member is attached to the peripheral frame at the exterior side of the arc shape thereof.

4. An obstructing device according to claim 2, wherein said arc shape of said frame extends from said proximal end to said distal end thereof and wherein said arc shape of said clipping arm extends from a proximal end to a distal end.

5. An obstructing device according to claim 1, wherein said tubular member is elongate and has an axis and wherein said attachment element is elongate and has an axis and wherein said elongate hollow tubular member is attached to said peripheral frame such that when said elongate attachment element is mounted on a cusp so that said axis points in a downstream direction, said elongate hollow tubular member also points in a downstream direction, and wherein said hollow tubular member comprises:
a proximal opening at its proximal end;
a substantially tubular surface extending distally from said proximal opening;
a distal end, being either closed or comprising a small orifice.

6. (canceled)

7. An obstructing device according to claim 1, wherein in said peripheral frame, each of said side arms being attached to said distal end and proximal end and said clipping arm does not extend to said distal end.

8-9. (canceled)

10. The obstructing device according to claim 1, wherein the clipping arm comprises a plurality of protrusions adapted to engage cusp tissue and extending in a direction away from said preferred bending plane.

11. The obstructing device according to claim 1, wherein each one of the side arms comprises a plurality of protrusions adapted to engage cusp tissue and extending in a direction towards the other side arm.

12. (canceled)

13. The obstructing device according to claim 1, wherein the hollow tubular member comprises a membrane.

14. The obstructing device according to claim 13, wherein the membrane is self-expandable.

15. (canceled)

16. The obstructing device according to claim 1, wherein the hollow tubular member tappers distally in a downstream direction.

17. The obstructing device according to claim 1, wherein the peripheral frame comprises one or more apertures.

18. The obstructing device according to claim 2, wherein the attachment element is made of a super-elastic or shape memory material.

19. The obstructing device according to claim 1, wherein the attachment element comprises connecting elements configured to engage cusp material.

20-22. (canceled)

23. A delivery system for delivering an obstructing device for being mounted on a native cusp or leaflet, said delivery system comprising:
a body;
an obstructing device according to claim 1; and
a capsule at a distal end of said body, the capsule comprising:
a recess for receiving said obstructing device;
a distal holder for holding a distal part of said obstructing device;
a proximal holder for holding a proximal side of said obstructing device;
wherein said one of said proximal and said distal holders is movable to two positions, a first position where a section of said obstructing device is exposed, allowing part of said obstructing device to elastically extend away from said capsule and a second position where the proximal or distal end of the obstructing device is released from being held.

24. A delivery system according to claim 23, comprising a spring configured to push said obstructing device away from said capsule when said obstructing device is released at both a distal end and a proximal end thereof.

25. A delivery system according to claim 23, comprising an obstructing device in said recess, wherein said obstructing device comprises a curved frame with a curved resting state, wherein said capsule holds said frame to prevent said curved frame from being at said curved resting state.

26-29. (canceled)

30. A method for implanting an obstructing device on a heart valve cusp or leaflet, comprising:
advancing passing a delivery catheter system having an obstructing device mounted therein, until the distal portion of the catheter arrives near a target heart valve cusp or leaflet;
releasing a clipping arm of said obstructing device to extend radially away from said delivery catheter system and define a gap therebetween;
positioning said cusp or leaflet within said gap; and
further releasing at least a further part of said obstructing device comprising a frame with two side arms to elastically extend away from said delivery catheter system so that said gap is closed and said cusp or leaflet is trapped by between said clipping arm and said frame.

31. A method according to claim 30, further comprising releasing said obstructing device entirely from said delivery catheter system using an elastic recoil, wherein said frame is an elastically deformable frame and wherein said releasing comprises releasing said frame and said clipping arm, wherein said cusp or leaflet prevents said clipping arm from reaching a resting position on an opposite side of said cusp or leaflet.

32-34. (canceled)

35. A method according to claim 31, wherein said releasing comprises allowing said elastically deformable frame to reach a curved resting position, wherein said curved position is curved in a same direction as said clipping arm.

36. An obstructing device according to claim 2, wherein said peripheral frame and said central arm are curved in a same direction.

37. The obstructing device according to claim 13, wherein the membrane, is collapsible.

38. A delivery system for delivering an obstructing device for being mounted on a native cusp or leaflet, said delivery system comprising:
a body;
a capsule at a distal end of said body, the capsule comprising:
a recess for receiving an obstructing device;
a distal holder for holding a distal part of said obstructing device;
a proximal holder for holding a proximal side of said obstructing device;
wherein said one of said proximal and said distal holders is movable to two positions, a first position where a section of said obstructing device is exposed, allowing part of said obstructing device to elastically extend away from said capsule and a second position where the proximal or distal end of the obstructing device is released from being held.
